(19) United States
(12) Patent Application Publication
(10) Pub. No.: US 2025/0040914 A1
(43) Pub. Date: Feb. 6, 2025

(54) SYSTEMS AND METHODS FOR FOCUSED ULTRASOUND-ENABLED LIQUID BIOPSY

(71) Applicant: Washington University, St. Louis, MO (US)

(72) Inventors: Hong CHEN, St. Louis, MO (US); Zhongtao HU, St. Louis, MO (US); Lu XU, St. Louis, MO (US); Christopher PACIA, St. Louis, MO (US); Chih-Yen CHIEN, St. Louis, MO (US); Eric LEUTHARDT, St. Louis, MO (US)

(21) Appl. No.: 18/693,320

(22) PCT Filed: Sep. 26, 2022
(86) PCT No.: PCT/US2022/044718

§ 371 Date: Mar. 19, 2024

Related U.S. Application Data
(60) Provisional application No. 63/247,914, filed on Sep. 24, 2021.

(51) Int. Cl. A61B 10/00; A61B 5/00; C12Q 1/6851; C12Q 1/6886

(52) U.S. Cl. CPC A61B 10/0045; A61B 5/0051; C12Q 1/6851; C12Q 1/6886; A61B 2010/0077

(57) ABSTRACT

Devices, systems, and methods for controlling the operation of a focused ultrasound blood-brain barrier opening (FUS-BBBO) device using an individualized closed-loop feedback control method are disclosed. Devices, systems, and methods for performing transcranial cavitation localization using a time difference of arrival method combined with signal processing using differential cavitation are also disclosed. Methods for performing a liquid biopsy to diagnose a brain disorder using a FUS-BBBO method to enhance the release of biomarkers from the brain into CSF and blood are also disclosed.

### CROSS-REFERENCE TO RELATED APPLICATIONS

This application claims priority from U.S. Provisional Application Ser. No. 63/247,914 filed on Sep. 25, 2021, which is incorporated herein by reference in its entirety.

### STATEMENT REGARDING FEDERALLY SPONSORED RESEARCH OR DEVELOPMENT

This invention was made with government support under N00014-19-1-2335 awarded by the Office of Naval Research and EB030102 awarded by the National Institutes of Health. The government has certain rights in the invention.

### MATERIAL INCORPORATED-BY-REFERENCE

Not applicable.

### FIELD OF THE DISCLOSURE

The present disclosure generally relates to systems and methods for 3D passive transcranial cavitation detection. The present disclosure further relates to systems, devices, and methods for focused ultrasound-enabled brain-blood barrier opening (FUS-BBBO), as well as focused ultrasound-enabled liquid biopsy.

### BACKGROUND OF THE DISCLOSURE

Cavitation is a fundamental physical mechanism of various focused ultrasound (FUS)-mediated therapies in the brain. Accurately knowing the 3D location of cavitation in real-time can improve the treatment targeting accuracy and avoid off-target tissue damage.

Techniques that combine focused ultrasound (FUS) combined with microbubbles are gaining increased use for a variety of neurological applications that employ cavitation as the primary physical mechanism, such as FUS combined with microbubble-mediated blood-brain barrier (BBB) disruption for targeted and gene delivery, cavitation-enhanced non-thermal ablation, FUS-enabled liquid biopsy, and transcranial histotripsy. Real-time three-dimensional (3D) transcranial cavitation localization is critical in these applications to ensure precise targeting of the FUS and avoid off-target tissue damage. Transcranial cavitation localization faces major challenges presented by the skull, which induces significant phase aberration and amplitude attenuation to the transcranially detected cavitation signals.

Existing passive cavitation detection techniques typically use a single-element ultrasound sensor to detect the presence of cavitation by analyzing the spectral characteristics of the detected acoustic emissions. Although a single-element receiver is able to efficiently detect the presence of cavitation activity, the measurements obtained by a single-element receiver are insufficient to deduce the spatial localization of the cavitation.

Recently, 3D passive cavitation imaging using a hemispherical phased array combined with passive beamforming and computed tomography (CT)-based skull-specific aberration correction algorithms were developed for 3D imaging of microbubbles associated with FUS-mediated BBB disruption and transcranial histotripsy therapy. Existing methods of 3D passive cavitation imaging produce a spatial distribution of cavitation activity but require expensive customized phased arrays with 256 elements or more, and the phased-array data must be processed using complicated and time-consuming computation algorithms.

The blood-brain barrier (BBB) is a natural barrier in the brain that prevents most systemically administrated therapeutic agents from reaching the brain parenchyma. Focused ultrasound (FUS) in combination with intravenously injected microbubbles for blood-brain barrier opening (FUS-BBBO) has been established as a promising technique for delivering therapeutic agents to a targeted brain region without invasive surgery. Its safety and efficacy have been demonstrated in small animals, large animals, and humans. A relatively narrow window of acoustic energy within which FUS-BBBO can be safely and effectively performed has been identified. Insufficient FUS energy yields limited BBB opening, while excessive FUS energy potentially leads to vascular disruption and permanent tissue damage. Cavitation is the fundamental physical mechanism of FUS-BBBO. Depending on the acoustic pressure, microbubble cavitation can range from stable cavitation (SC) to inertial cavitation (IC). Microbubbles undergo sustained, low-amplitude volumetric oscillation (SC) at low acoustic pressures, which could increase the BBB permeability without causing any vascular damage. Microbubbles typically expand to large sizes and collapse violently (IC) at high acoustic pressures, which increase BBB permeability but may induce vascular disruption. In order to maintain FUS exposure within a safe and effective window, passive cavitation detection (PCD)-based feedback control algorithms have been proposed for real-time monitoring of cavitation and providing feedback control of the FUS sonication pressure.

One existing feedback control algorithm to achieve safe FUS-BBBO included increasing the sonication pressure until ultra-harmonic signals or sub-harmonic signals from microbubble emissions were detected (ramping-up phase), decreasing the acoustic pressure to 50% of the final ramped-up value, and maintaining acoustic pressure at this level for subsequent treatments in an open-loop fashion (i.e. maintaining phase). This approach considered the individual differences in the detected cavitation signals as the threshold was defined based on calibration performed for an individual subject during the ramping-up phase. The individual differences in the detected cavitation signals could arise from several factors, including variation in the in situ acoustic pressure in the brain due to changes in skull thickness and the incident angle of the FUS beam; variation in microbubble concentration and size distribution for each injection; and the heterogeneous spatial distribution of the microbubbles in the brain due to differences in vascular density, vessel size, and blood flow. However, there are two potential limitations of this existing feedback control algorithm: the pressure ramping-up phase requires the pressure overshoot to reach the threshold and then decrease to a safe level, which carries the risk of causing tissue damage. Further, the maintaining phase uses an open-loop approach that maintains the acoustic pressure at a fixed value. To minimize overexposure in the ramping-up phase, other existing feedback control algorithms modulated the acoustic power level until the mean harmonic signal reached a target between 6-7.5 dB above the noise level detected before microbubble injection and then fixed to the average pressure level that resulted in this target range for the remaining sonication, or used a relative spectrum defined as the ratio of the instantaneous signal power spectrum after microbubble injection and the corresponding baseline power spectrum before microbubble injection to define sonication levels.

Several closed-loop feedback control algorithms have been proposed for FUS-BBBO. One existing closed-loop algorithm uses an adaptive proportional-integral controller for drug delivery across the BBB in a rat glioma model that monitors the cavitation emissions throughout the experiment and adjusts the ultrasound pressure level based on the previous state of the controller and a targeted cavitation level (TCL). In these existing methods, TCL was defined as the maximum harmonic emission level achieved without broadband detection based on prior experiments, and the same TCL was used for all subjects. Another existing closed-loop algorithm regulated the sonication pressure for each pulse to maintain the cavitation level within a predefined range. An additional closed-loop algorithm implemented a closed-loop nonlinear state controller to control the acoustic exposure level based on passive cavitation imaging that enabled spatially specific measurement of cavitation activity for spatial-selective feedback control of FUS-BBBO. However, passive cavitation imaging requires the use of a customized ultrasound imaging system coupled with an advanced beamforming technique, which limits the broad application of this method in FUS-BBBO. These existing closed-loop feedback control algorithms control cavitation activity in real-time in a closed-loop fashion but apply the same predefined TCL to all subjects without considering individual differences in their baseline cavitation signals.

### SUMMARY OF THE DISCLOSURE

In various aspects, systems, devices, and methods for performing a liquid biopsy to diagnose a brain disorder of a subject are disclosed. In various other aspects, devices, systems, and methods for controlling the operation of a focused ultrasound blood-brain barrier opening (FUS-BBBO) device are disclosed herein. In various additional aspects, systems, devices, and methods of transcranially localizing cavitations within a skull of a subject are disclosed.

In one aspect, a method for performing a liquid biopsy to diagnose a brain disorder of a subject is disclosed that includes injecting an amount of microbubbles into the subject, opening a blood-brain barrier of the subject using a focused ultrasound blood-brain barrier opening (FUS-BBBO) device to release at least one biomarker from a brain of the subject into blood and CSF of the subject, obtaining a biological sample comprising the at least one biomarker, and diagnosing the brain disorder based on the at least one biomarker isolated from the biological sample. The biological sample may be a blood sample or a CSF sample from the subject. In some aspects, opening the blood-brain barrier using the FUS-BBBO device further comprises sonicating the brain of the subject at a baseline sonication pressure and detecting a baseline stable cavitation level from the subject using the FUS-BBBO device. The baseline stable cavitation levels fall above signal noise and below a stable cavitation level sufficient to induce BBBO, and the subject is injected with the amount of microbubbles prior to sonication. In some aspects, opening the blood-brain barrier using the FUS-BBBO device further comprises sonicating the subject at a series of stepwise increasing sonication pressures and detecting a corresponding series of cavitation levels until a target cavitation level (TCL) is detected. In some aspects, opening the blood-brain barrier using the FUS-BBBO device further comprises continuously sonicating the subject to maintain the TCL to induce BBBO in the subject. The target cavitation level is a predetermined amount above the baseline stable cavitation level. In some aspects, detecting the baseline cavitation levels, the series of cavitation levels, and the target cavitation levels further comprises detecting microbubble cavitation signals, wherein the microbubble cavitation signals are produced by microbubbles in response to sonication by the FUS-BBBO device. In some aspects, the microbubble cavitation signals are processed using a Fast-Fourier transform (FFT) algorithm to produce the baseline cavitation levels, cavitation levels, and TCL. In some aspects, the target cavitation level may be one of 0.5 dB, 1 dB, 2 dB, 3 dB, or 4 dB above the baseline stable cavitation level.

In another aspect, a system to control the operation of a focused ultrasound blood-brain barrier opening (FUS-BBBO) device configured to perform FUS-BBBO on a subject is disclosed. The system includes a computing device operatively coupled to the FUS-BBBO device and a computing device comprising at least one processor. The at least one processor is configured to sonicate the brain of the subject at a baseline sonication pressure and detect a baseline stable cavitation level from the subject using the FUS-BBBO device. The baseline stable cavitation levels fall above signal noise and below a stable cavitation level sufficient to induce BBBO. The subject is injected with microbubbles prior to sonication. The at least one processor is further configured to sonicate the subject at a series of stepwise increasing sonication pressures and detecting a corresponding series of cavitation levels until a target cavitation level (TCL) is detected. The target cavitation level is a predetermined amount above the baseline stable cavitation level. The at least one processor is further configured to continuously sonicate the subject to maintain the TCL to induce BBBO in the subject. In some aspects, the system further comprises at least one passive cavitation detection (PCD) transducer to detect the baseline cavitation levels, the series of cavitation levels, and the target cavitation levels. In some aspects, detecting the baseline cavitation levels, the series of cavitation levels, and the target cavitation levels further comprises detecting microbubble cavitation signals, wherein the microbubble cavitation signals are produced by microbubbles in response to sonication by the FUS-BBBO device. In some aspects, the microbubble cavitation signals are processed using a Fast-Fourier transform (FFT) algorithm to produce the baseline cavitation levels, cavitation levels, and TCL. In some aspects, the microbubble cavitation signals comprise acoustic signals with a frequency within a bandwidth of a center frequency of the PCD transducer. In some aspects, the target cavitation level comprises one of 0.5 dB, 1 dB, 2 dB, 3 dB, or 4 dB above the baseline stable cavitation level.

In an additional aspect, a method of performing FUS-BBBO on a subject is described. The method includes injecting the subject with microbubbles, sonicating the subject at a baseline sonication pressure, and detecting a baseline stable cavitation level from the subject after injection of microbubbles using the FUS-BBBO device. The baseline stable cavitation levels fall above signal noise and below a stable cavitation level sufficient to induce BBBO. The method further includes sonicating the subject at a series of stepwise increasing sonication pressures and detecting a corresponding series of cavitation levels until a target cavitation level (TCL) is detected. The target cavitation level is a predetermined amount above the baseline stable cavitation level. The method further includes continuously sonicating the subject to maintain the TCL to induce BBBO in the subject. In some aspects, detection of baseline cavitation levels, the series of cavitation levels, and the target cavitation levels are performed using at least one passive cavitation detection (PCD) transducer. In some aspects, detecting the baseline cavitation levels, the series of cavitation levels, and the target cavitation levels further comprises detecting microbubble cavitation signals, wherein the microbubble cavitation signals are produced by microbubbles in response to sonication by the FUS-BBBO device. In some aspects, the microbubble cavitation signals are processed using a Fast-Fourier transform (FFT) algorithm to produce the baseline cavitation levels, cavitation levels, and TCL. In some aspects, the microbubble cavitation signals comprise acoustic signals with a frequency within a bandwidth of a center frequency of the PCD transducer. In some aspects, the target cavitation level comprises one of 0.5 dB, 1 dB, 2 dB, 3 dB, or 4 dB above the baseline stable cavitation level.

In another additional aspect, a device for transcranial cavitation localization in a subject is disclosed. The device includes four acoustic sensors to detect cavitation signals within a skull of the subject. The four acoustic sensors comprise S1, S2, S3, and S4. The four acoustic sensors are positioned in a fixed pattern configured to conform to the skull of the subject. The device further includes a focused ultrasound (FUS) transducer to sonicate a volume of interest within the skull of the subject, and a computing device comprising at least one processor. The at least one processor is configured to sonicate the volume of interest using the FUS transducer, receive a plurality of cavitation signals from within the skull of the subject at the four acoustic sensors, wherein the subject is injected with microbubbles, identify at least three time delays based on the plurality of cavitation signals, and localize the cavitation signal source based on the at least three time delays. The at least three time delays include a difference in an arrival time of a cavitation signal at one of the acoustic sensors S1, S2, S3, and S4 relative to one of the remaining acoustic sensors. In some aspects, the four acoustic sensors are positioned in a hemispherical pattern. In some aspects, the four acoustic sensors are positioned with three acoustic sensors arranged along a circumference of a circle and one acoustic sensor positioned within the circle and perpendicularly offset from the plane of the circle. In some aspects, each time delay of the at least three time delays is identified based on the maximum cross-correlation of a first sample of cavitation signals detected at a first acoustic detector and a second sample of cavitation signals detected at a second acoustic detector. In some aspects, the cavitation signal source is localized using a time difference of arrival (TDOA) method.

Other objects and features will be in part apparent and in part pointed out hereinafter.

### DESCRIPTION OF THE DRAWINGS

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5A is an image of a four-sensor network used in experiments described herein.

FIG. 5B is an image showing the sensor network of FIG. 5A positioned around a piece of ex vivo human skull.

FIG. 5C is a schematic diagram of an experimental setup of the four-sensor network to locate the microbubbles formed within a tube of water sonicated by a single FUS transducer.

FIG. 6 is a schematic of an experimental setup as used in experiments described herein including the sensor network of FIG. 5A positioned around a piece of ex vivo human skull as shown in FIG. 5B along with additional devices and elements.

FIG. 7A is a graph showing a time-domain comparison of signals obtained with and without the skull effects.

FIG. 7B is a graph showing a frequency-domain comparison of signals obtained with and without the skull effects.

FIG. 8A is a graph showing a time history of signals obtained with and without microbubbles.

FIG. 8B is a graph showing a time history of the post-subtraction difference between the signals with and without microbubbles shown in FIG. 8A.

FIG. 9 is a series of graphs showing the time histories of signals measured by the four sensors of the network illustrated in FIG. 5A, showing the time delay between sensor pairs.

FIG. 10A is a B-mode ultrasound image showing the setting position of the cavitation.

FIG. 10B is a graph showing the cavitation position obtained using the disclosed DCL method.

FIG. 10C is the B-mode image of FIG. 10A overlaid with the DCL cavitation position of FIG. 10B.

FIG. 11A is an image showing the sensor network of FIG. 5A positioned with sensor S2 at a P1 position corresponding to the occipital crest of the skull.

FIG. 11B is an image showing the sensor network of FIG. 5A positioned with sensor S2 at a P2 position corresponding to the frontal crest of the skull.

FIG. 11C is an image showing the sensor network of FIG. 5A positioned with sensor S2 at a P3 position corresponding to the position off of the occipital and frontal crests of the skull.

FIG. 12A is a graph showing sensor locations and cavitation locations as controlled by a 3D positioner using the experimental setup of FIG. 6.

FIG. 12B is a bar plot summarizing the accuracy of the disclosed DCL method without and with skull (averaged over all the 19 locations, with 10 replicates for each location); the bar plots denote the mean values with standard deviation.

FIG. 12C is a graph showing the cavitation source positions and estimated locations by the disclosed DCL method at the xy-plane with the skull.

FIG. 12D is a graph showing the cavitation source positions and estimated locations by the disclosed DCL method at the xz-plane with the skull.

FIG. 12E is a graph showing the cavitation source positions and estimated locations by the disclosed DCL method at the xy-plane without the skull.

FIG. 12F is a graph showing the cavitation source positions and estimated locations by the disclosed DCL method at the xz-plane without the skull.

FIG. 13A is a bar graph showing the localization accuracy of the cavitation source with and without skull as a function of source locations along the x-axis; 19 locations total, and 30 replicates for each location.

FIG. 13B is a bar graph showing the localization accuracy of the cavitation source with and without skull as a function of source locations along the y-axis; 19 locations total, and 30 replicates for each location.

FIG. 13C is a bar graph showing the localization accuracy of the cavitation source with and without skull as a function of source locations along the z-axis; 19 locations total, and 30 replicates for each location.

FIG. 14 is a bar graph showing the accuracy of transcranial localization for the different sensor network orientations as illustrated in FIGS. 11A (P1), 11B (P2), and 11C (P3) using the disclosed DCL method averaged over 30 replicates for each sensor position.

FIG. 15 is a bar graph showing the accuracy of transcranial localization as a function of FUS peak negative pressures for a cavitation source located at the geometric focus of the sensor network using the disclosed DCL method, averaged over 30 replicates for each case.

FIG. 16 is a bar graph showing the accuracy of transcranial localization as a function of source cycles for a cavitation source located at the geometric focus of the sensor network averaged over 30 replicates for each case.

FIG. 17A is an image of a four-sensor network used in experiments described herein.

FIG. 17B is an image showing the sensor network of FIG. 17A positioned around a piece of ex vivo human skull.

FIG. 17C is a schematic diagram of an experimental setup of the four-sensor network to locate the microbubbles formed within a tube of water sonicated by a single FUS transducer.

FIG. 18A is a graph showing sensor locations and cavitation locations as controlled by a 3D positioner using the experimental setup of FIG. 17C.

FIG. 18B is a bar plot summarizing the positional error of the microbubble positions determined using the disclosed DCL method without and with the skull; the bar plots denote mean values with standard deviations.

FIG. 18C is a graph summarizing the cavitation source positions (darker dot) and locations estimated using the disclosed DCL method (lighter dot) at the xy-plane within the skull.

FIG. 18D is a graph summarizing the cavitation source positions (darker dot) and locations estimated using the disclosed DCL method (lighter dot) at the xz-plane within the skull.

FIG. 19 is a schematic Illustration of a feedback-controlled FUS system. The experiment setup was composed of three parts: (1) Transmission: FUS transducer, function generator, and power amplifier. (2) Receiving: PCD, pre-amplifier, computer-based oscilloscope, and Picoscope. (3) Feedback control: a customized MATLAB-based graphic user interface (GUI) implementing the feedback control algorithm.

FIG. 20 is a schematic Illustration of a feedback control algorithm. Microbubble infusion was started 15 seconds before FUS sonication and lasted until the end of FUS sonication. During FUS sonication with microbubbles infusion, cavitation was monitored by PCD in real-time. The baseline cavitation level for each mouse was defined by 10 repeated PCD measurements acquired during dummy FUS sonication. Once TCL was defined (i.e., 0.5 dB, 1 dB, 2 dB, 3 dB, or 4 dB above the baseline SC level), FUS sonication was performed with the feedback control algorithm in a two-phase process: the pressure ramping-up phase to have SC level reach TCL and maintaining phase to keep SC level within the target range (i.e., TCL±tolerance range). The tolerance range was set to ±0.4 dB to tolerate SC level fluctuation.

FIG. 21 contains a series of graphs illustrating a method of determining the TCL of the disclosed individualized feedback control algorithm at each target level (i.e., 0.5 dB, 1 dB, 2 dB, 3 dB, or 4 dB). Each group included five mice and each circular point represents the result obtained from each mouse. Box limits, 25 and 75 percentiles; whiskers, 5 and 95 percentiles, centerline, median).

FIG. 22A contains a series of graphs summarizing the measured SC levels as a function of time at different TCLs. Each gray scale shade represents the SC level obtained from each mouse. The solid line on the right of the second row represents the average SC level for each TCL group (i.e., 0.5 dB, 1 dB, 2 dB, 3 dB, or 4 dB above baseline SC level).

FIG. 22B is a graph of the percentage of good burst rates at each TCL to illustrate the stability of the feedback control algorithm at each TCL. The box plot shows the median and standard deviation. Each circular point represents the result obtained from each mouse.

FIG. 22C is a graph summarizing the average IC levels measured over time at each TCL.

FIG. 22D is a graph summarizing the IC probability at different TCLs.

FIG. 23A contains representative photographs and corresponding fluorescence images at five TCLs (i.e. 0.5 dB, 1 dB, 2 dB, 3 dB, and 4 dB). The gray scale intensity bar on the right indicates the fluorescence intensity.

FIG. 23B is a graph summarizing the normalized fluorescence intensity at each TCL.

FIG. 23C is a graph summarizing the drug (i.e., Evans blue) delivery area at each TCL. FUS-BBBD delivery efficiency and drug delivery area were observed to be increased when the TCL was increased from 0.5 dB to 3 dB and slightly decreased at 4 dB. Each circular point represents the result obtained from each mouse. *P<0.05, **P<0.01, and ***P<0.001

FIG. 24A contains representative H&E staining images of the FUS-treated side of the brainstem at each TCL.

FIG. 24B is a graph comparing the hemorrhage area of each TCL to the corresponding contralateral side. *P<0.05 FIG. 25A is a graph of a representative SC level of the feedback control algorithm used in a clinical trial.

FIG. 25B is a graph showing a correlation between fluorescence intensity and the SC dose of the ramping-up phase.

FIG. 25C is a graph showing a correlation between fluorescence intensity and the SC dose of the maintaining phase.

FIG. 26A is a schematic of the hardware setup for MRI-guided sonobiopsy in mice. The FUS transducer was coupled with the mouse head using ultrasound gel and a bladder filled with degassed water.

FIG. 26B is a set of MRI images. Contrast-enhanced (CE) T1-weighted MRI scans were acquired before FUS to quantify the tumor volume (bottom left brighter spot). Post-FUS MRI scans confirmed FUS-induced BBB disruption (bottom right bright area) as an increase in CE volume.

FIG. 26C is a graph showing that FUS significantly increased the CE volume (n=19, p=0.0000038; ****p<0.0001; paired samples Wilcoxon signed rank test) from 24.59±13.21 mm³ to 46.09±20.44 mm³. Black bars indicate mean.

FIG. 27A is a pair of two 1 D amplitude plots for blood LBx and sonobiopsy groups that demonstrate the detection of EGFRvIII in plasma for each representative subject. The line depicts the threshold fluorescence for identifying droplets with positive EGFRvIII expression.

FIG. 27B is a graph that shows the level in the sonobiopsy group (n=17; 19.06±24.74 copies/μL) was significantly greater (p=0.00089; ***p<0.001; unpaired two-sample Wilcoxon signed rank test) compared with the level in the blood LBx group (n=14; 0.02±0.08 copies/μL). Black bars indicate mean.

FIG. 27C is a pair of 1D amplitude plots for the detection of TERT C228T in plasma for each representative subject in the blood LBx and sonobiopsy groups. The line depicts the threshold fluorescence for identifying droplets with positive TERT C228T expression.

FIG. 27D is a graph that shows FUS significantly increased the levels of TERT C228T ctDNA in the plasma from 0.06±0.18 copies/μL in the blood LBx group (n=21) to 0.64±1.19 copies/μL in the sonobiopsy group (n=24; p=0.015; *p<0.01; unpaired two-sample Wilcoxon signed rank test). Black bars indicate mean.

FIG. 27E is a graph that shows, with ddPCR, sonobiopsy is more sensitive than blood LBx with a detection rate of 64.71% for EGFRvIII and 45.83% for TERT C228T compared with 7.14% and 14.29% for blood LBx, respectively. ND: not detected.

FIG. 28A is a representative H&E staining image for a subject treated with sonobiopsy. The arrow points to microhemorrhage in the tumor ROI.

FIG. 28B is a graph that shows the microhemorrhage density in the parenchyma after sonobiopsy (0.47±0.68 positive pixels/μm², n=5) was not significantly different compared with that after blood LBx (0.83±0.69 positive pixels/μm²; n=5, p=0.33; unpaired two-sample Wilcoxon signed rank test).

There was a nonsignificant increase in microhemorrhage occurrence in the tumor ROI after sonobiopsy (4.54±3.08 positive pixels/μm², n=5) compared with that after blood LBx (2.08±3.54 positive pixels/μm²; n=5, p=0.18; unpaired two-sample Wilcoxon signed rank test). Black bars indicate mean.

FIG. 28C is a representative TUNEL staining image for a subject treated with sonobiopsy that depicts an increased apoptotic signal in the tumor ROI. The arrow points to an apoptotic cell.

FIG. 28D is a graph that shows there was no significant difference in TUNEL density for the parenchyma between blood LBx (0.20×10^{-3±0.22}×10⁻³ positive cells/μm², n=5) and sonobiopsy (0.47×10^{-3±0.22}×10⁻³ positive cells/μm²; n=5, p=0.11; unpaired two-sample Wilcoxon signed rank test). There was no significant difference in TUNEL density for the tumor ROI between blood LBx (1.82×10^{-3±0.62}×10⁻³ positive cells/μm2, n=5) and sonobiopsy (1.97×10^{-3±1.22}×10⁻³ positive cells/μm²; n=5, p=0.73; unpaired two-sample Wilcoxon signed rank test). Black bars indicate mean.

FIG. 29A is an image of the hardware setup for MRI-guided sonobiopsy in pigs. The pig head was stabilized by the head supports. The MR-compatible motor enabled the translation of the FUS transducer to specific target locations.

FIG. 29B is an image that shows the placement of a pig in a sonobiopsy device.

FIG. 29C is a set of MRI images. CE T1-weighted MRI scan shows tumor volume (bottom left bright spot) and FUS-induced BBB disruption (bottom right bright area).

FIG. 29D is a graph that shows the CE volume significantly increased (n=6; p=0.031; *p<0.05; paired samples Wilcoxon signed rank test) from 348.70±358.02 mm³ to 799.50±501.19 mm³. Black bars indicate mean.

FIG. 30A is a pair of 1D amplitude plots for EGFRvIII detection in plasma for each subject.

FIG. 30B is a graph that shows sonobiopsy significantly increased plasma levels of EGFRvIII ctDNA (n=7; p=0.016; *p<0.05; paired samples Wilcoxon signed rank test) from 13.69±28.62 copies/mL to 3697.54±3780.61 copies/mL. Black bars indicate mean.

FIG. 30C is a pair of 1D amplitude plots for TERT C228T detection in plasma for each subject.

FIG. 30D is a graph that shows sonobiopsy significantly increased the plasma levels of TERT C228T ctDNA (n=10; p=0.022; *p<0.05; paired samples Wilcoxon signed rank test) from 13.07±23.08 copies/mL to 112.25±150.75 copies/mL. Black bars indicate mean.

FIG. 30E is a graph that shows, with ddPCR, sonobiopsy is more sensitive than blood LBx with a detection rate of 100% for EGFRvIII and 71.43% for TERT C228T compared with 28.57% and 42.86% for blood LBx, respectively. ND: not detected.

FIG. 31A is an image of a representative horizontal slice with H&E staining. The microhemorrhage occurs in some cases near the edge of the tumor (arrows).

FIG. 31B is a graph that shows microhemorrhage density was not significantly different between parenchyma (0.33±0.13 positive pixels/μm², n=4) and tumor (1.28±0.79 positive cells/μm², n=4, p=0.20; unpaired two-sample Wilcoxon signed rank test). Black bars indicate mean.

FIG. 31C is an image of a representative TUNEL staining that depicts the apoptotic cells (arrows).

FIG. 31D is a graph that shows there was no significant difference between the TUNEL density in the tumor (110.40×10^{-4±112.25}×10⁻⁴ positive cells/μm², n=4) compared with that in the parenchyma (51.34×10^{-4±56.12}×10⁻⁴ positive cells/μm²; (n=4, p=0.55; unpaired two-sample Wilcoxon signed rank test). Black bars indicate mean.

Those of skill in the art will understand that the drawings, described below, are for illustrative purposes only. The drawings are not intended to limit the scope of the present teachings in any way.

### DETAILED DESCRIPTION

Focused ultrasound in combination with microbubble-induced blood-brain barrier opening (FUS-BBBO) is a promising strategy for noninvasive and localized brain drug delivery, with a growing number of clinical studies currently ongoing. In addition to delivering drugs, low-intensity FUS sonication of the brain may be used to enhance the release of brain disease biomarkers into the blood and CSF to enable the noninvasive and reliable diagnosis of brain diseases. FUS sonication of the brain can generate mechanical and thermal effects in the brain that enhance the release of brain disease biomarkers into the blood and CSF.

### Method of Microbubble Cavitation-Assisted Liquid Biopsy of Brain Disorders

In various aspects, a method of performing a liquid biopsy to diagnose a brain disorder is disclosed herein. As described above, FUS-BBBO devices and methods enhance the release of biomarkers from the brain into the blood and CSF of a subject, thereby increasing the concentrations of the biomarkers to levels that are more readily detectable using various biomarker assays and assay methods.

In various aspects, the liquid biopsy method includes injecting an amount of microbubbles into a subject followed by opening the BBB of the subject using the FUS-BBBO methods and closed-loop feedback control of microbubble-induced cavitation as described herein.

In various aspects, after inducing BBB opening using the FUS-BBBO devices and methods, the method further includes collecting a biological sample from the subject containing the biomarkers. Non-limiting examples of suitable biological samples include a blood sample or a CSF sample. In various aspects, the blood samples and CSF samples are collected using any suitable existing method without limitation.

In various aspects, the liquid biopsy method further includes diagnosing the brain disorder based on one or more biomarkers isolated from the biological sample. Any suitable existing assay system, device, and method may be used to isolate and analyze the one or more biomarkers without limitation. In various aspects, the biomarker includes any suitable biomarker known to be indicative of a brain disorder including, but not limited to, cytokines, cells, cell-free DNA, RNA, proteins such as beta-amyloid proteins, exosomes, and any combination thereof. Non-limiting examples of brain disorders that may be diagnosed using the disclosed liquid biopsy method include brain cancer, Alzheimer's Disease, Parkinson's, and any other suitable brain disorder without limitation.

In various aspects, the efficacy of the disclosed liquid biopsy method is enhanced by reliably safe and effective opening of the BBB barrier using FUS-BBBO systems, devices, and methods as described herein. In one aspect, the microbubble cavitation is safely and reliably controlled using an individualized closed-loop feedback method that accounts for individual differences in a subject's morphology, the coupling of the FUS-BBBO sonication and cavitation detection elements to the cranium of the subject, and variations of microbubble composition and concentration for each individual and/or treatment.

### Methods of Individualized Closed-loop Feedback Control of Microbubble Cavitation

In various aspects, systems, devices, and methods for individualized closed-loop feedback control of microbubble cavitation for safe and reliable FUS-BBBO are disclosed herein. In various aspects, the disclosed FUS-BBBO feedback control method defines a target cavitation level (TCL) based on the baseline stable cavitation (SC) level for an individual subject with “dummy” FUS sonication. The dummy FUS sonication applies FUS at the targeted brain location at a low acoustic pressure for a short duration in the presence of microbubbles to define the baseline cavitation level that takes into consideration the individual differences in the detected cavitation emissions. FUS-BBBO is then conducted using two sonication phases: a ramping-up phase to reach a final phase that achieves the TCL and continuing to sonicate at this final phase to maintain the SC level at TCL. As described in the examples below, evaluations performed in wild-type mice demonstrated that the disclosed FUS-BBBO feedback control method achieved reliable and damage-free trans-BBB delivery of a model drug at selected TCL levels.

As described in the examples below, the individualized closed-loop feedback control method as disclosed herein achieved reliable and safe FUS-BBBO. The disclosed control method defines the TCL based on the baseline SC level acquired for an individual subject and thereby avoided overexposure to FUS during sonication of the subject associated with FUS-BBBO. As further described in the Examples below, the drug delivery outcome increased as the TCL increased from 0.5 dB to 2 dB above the baseline SC level without causing vascular damage; Increasing the TCL above 2 dB increased the probability of tissue damage.

Without being limited to any particular theory, FUS-BBBO is influenced by interactions among at least several factors including, but not limited to, the ultrasound energy delivered to the region, the concentration and structure of microbubbles delivered to the region, and individual cerebral vasculature morphologies. In various aspects, the disclosed control method defines the TCL based on cavitation signals generated by FUS sonication at low pressure for a period ranging from about 2 seconds to about 10 seconds or more. As described in the examples below, the TCL used for the mice for FUS-BBBO was based on about 5 seconds of cavitation signals generated by FUS sonication at a low pressure of 0.2 MPa as measured in water, corresponding to an estimated in situ acoustic pressure of about 0.16 MPa, assuming a mouse skull attenuation of about 18%.

In various aspects, the dummy sonication level used to evaluate an individual TCL is below the exposure energy needed to induce BBB opening. Without being limited to any particular theory, the evaluation of an individual TCL as described herein simultaneously accounts for individual variations in the delivery of FUS, the concentration and structure of microbubbles delivered to the region, and the morphology of the cerebral vasculature of the individual subject. Without being limited to any particular theory, the acoustic emissions detected with the dummy sonication used to evaluate the individual TCL may be influenced by one or more factors including but not limited to: individual differences in the skull thicknesses and incident angle of the FUS beam, which affects the in situ acoustic pressure and the skull-reflected FUS signals detected by the PCD; variations in the concentration and size distribution of injected microbubbles; and 3) heterogeneity in the spatial distribution of microbubbles near the BBB due to variations in vascular density, vessel size, and blood flow.

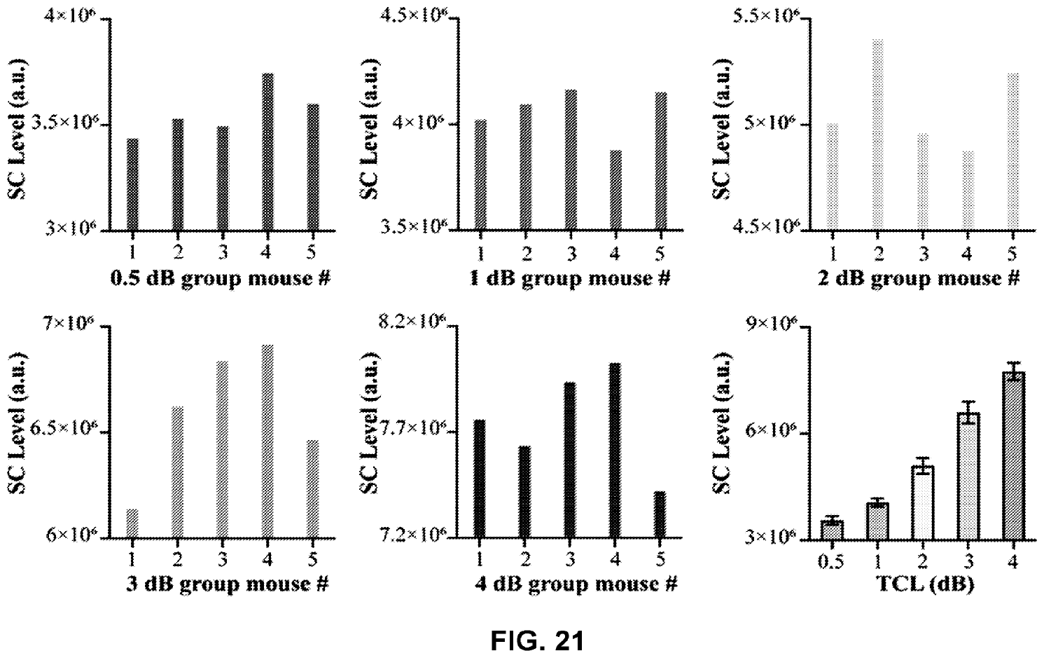

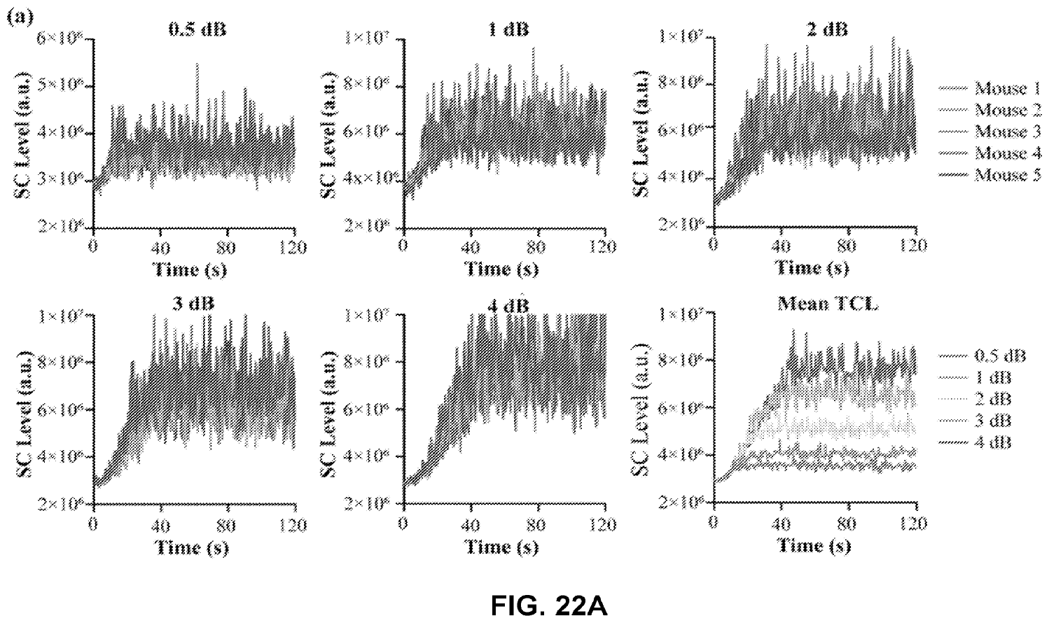

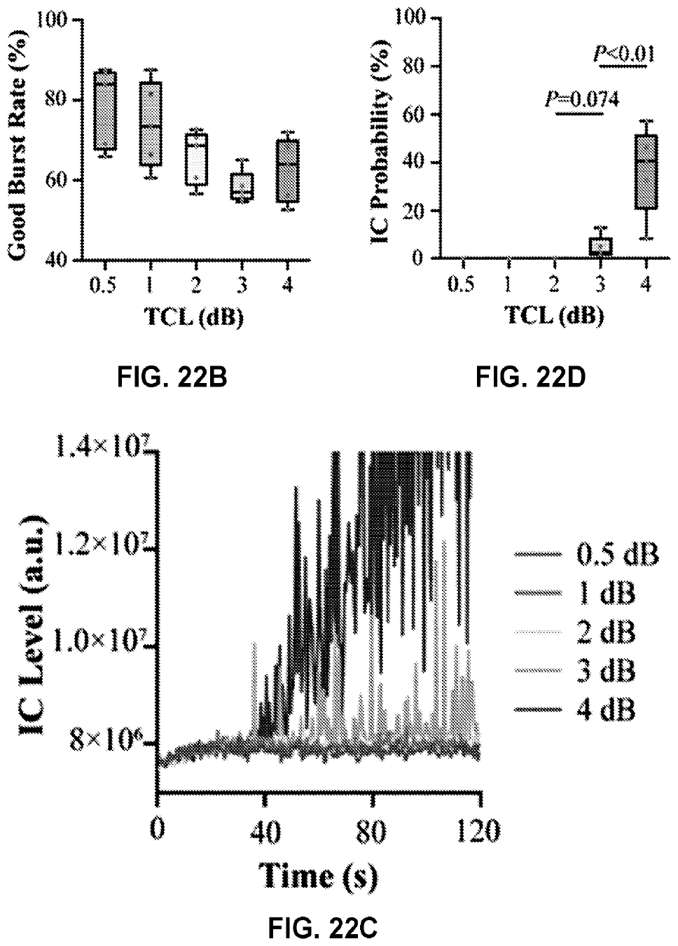

As described in the examples below, experimental data detected individual variations in the baseline SC level, as summarized in FIG. 21. Due to differences in the baseline SC level of each subject, the targeted absolute SC level of each subject was different (FIG. 21, lower right graph). The closed-loop feedback control method disclosed herein maintained the SC level at the TCL with high stability. As described in the examples below, the disclosed closed-loop feedback control method achieved stability, as quantified by a good burst rate, of 58.2-78.6% for TCL ranging from 0.5-4 dB (FIG. 22B), a stability level that is comparable to the best existing control methods.

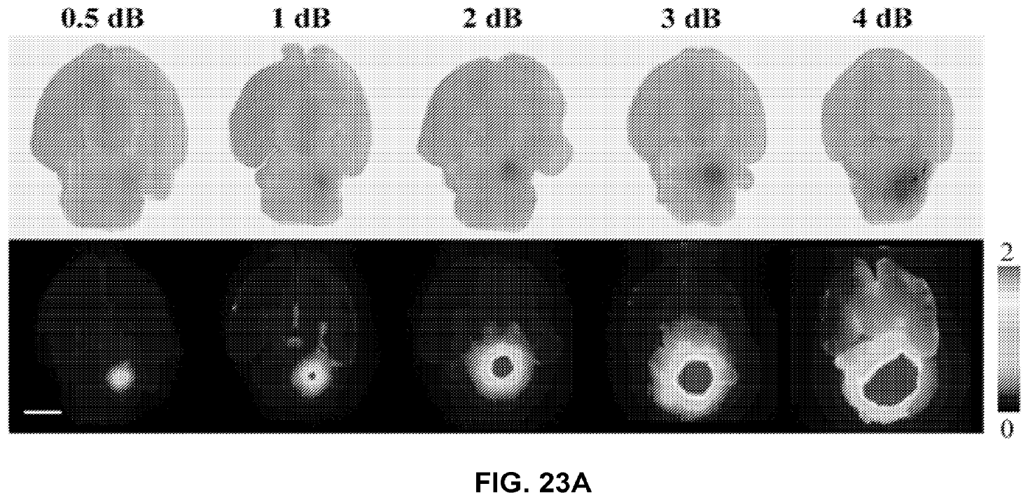

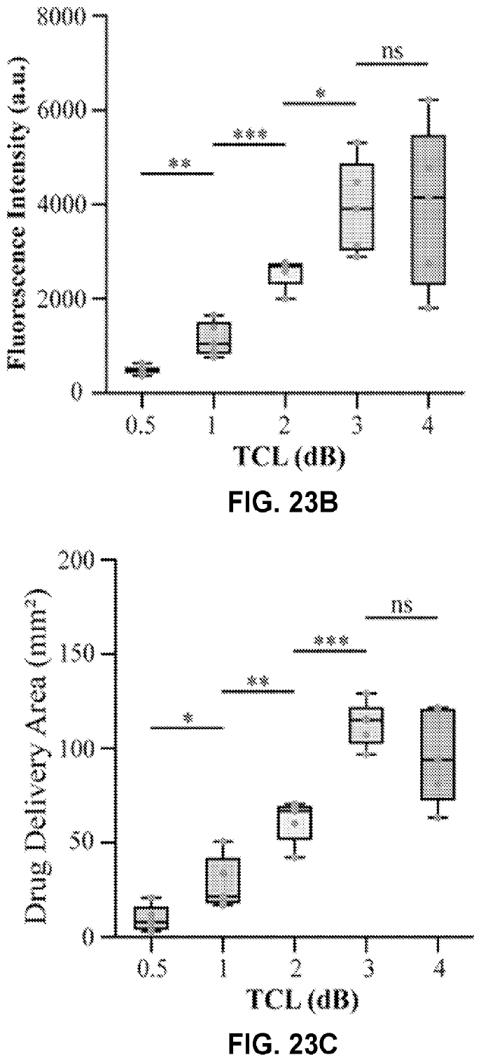

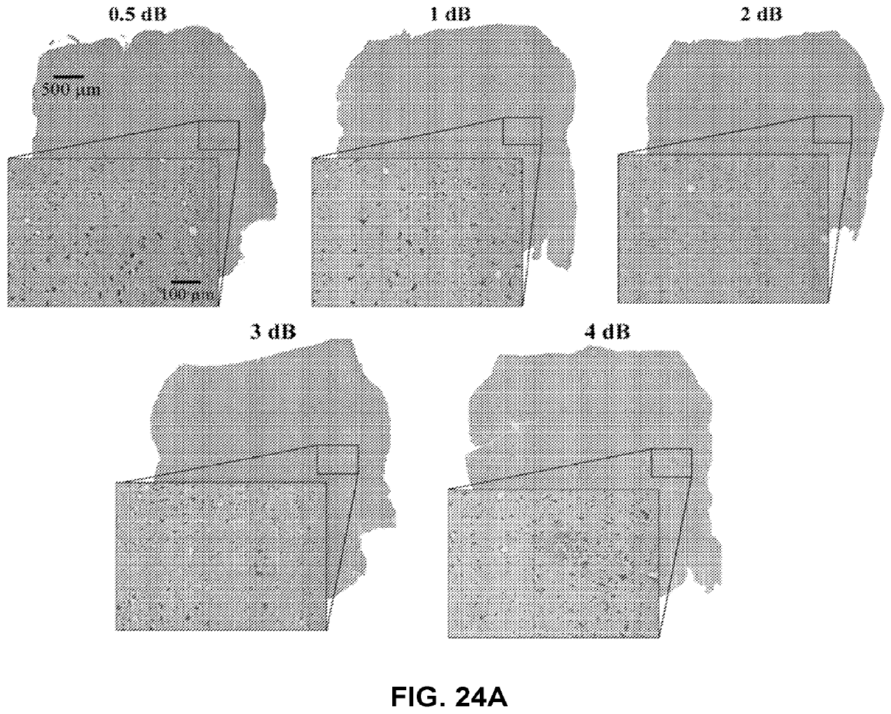

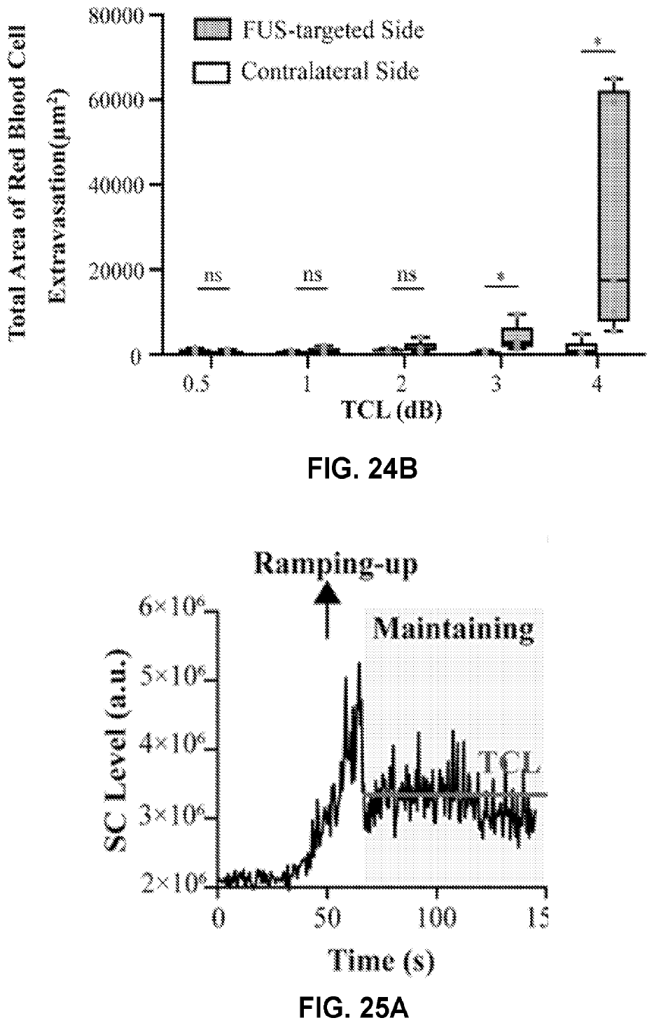

In various aspects, the selection of an optimal TCL needs to consider the stability of the feedback controller in addition to the FUS-BBBO delivery outcome and safety. As described in the examples, no significant difference was detected among the good burst rates of the five groups, but increasing TCL was observed to be associated with a trend of decreasing in the good burst rate (FIG. 22B), indicating decreasing stability/controllability among cavitation events. In addition, increasing TCL within the range of 0.5 dB to 3 dB induced an approximately linear increase in the Evans blue fluorescence intensity (FIG. 23B) indicating a more effective opening of the BBB and associated delivery of circulating compounds into the brain. However, higher TCLs were also associated with a higher probability of the presence of IC (FIG. 22D) and vascular damage (FIG. 24B). A TCL of 2 dB was identified as the optimal level for efficient and safe FUS-BBBO based on the relationships of TCL to the probability of IC and vascular damage described above.

In some aspects, the disclosed feedback control method may further include monitoring IC and modulating sonication pressure based at least in part on changes in the detected IC and/or estimated probability of IC. In one aspect, the feedback control algorithm may decrease the sonication pressure when IC is detected in order to avoid tissue damage.

In various aspects, the disclosed closed-loop feedback control method may be integrated into the operation of any suitable FUS-BBBO device or system without limitation. In various aspects, the disclosed control method may be implemented in the form of instructions executable on at least one processor of a computing device, described in additional detail below. In some aspects, the at least one processor resides on at least one computing device of a suitable FUS-BBBO device or system. In other aspects, the at least one processor resides on a separate computing device of a separate FUS-BBBO control system operatively coupled in communication with a suitable FUS-BBBO system or device.

### Transcranial Cavitation Detection Systems and Methods

Existing techniques for 3D passive transcranial cavitation detection require the use of expensive and complicated hemispherical phased arrays. In various aspects, devices and methods for 3D passive transcranial cavitation that make use of a small number of sensors (e.g, four) for transcranial 3D localization of cavitation are disclosed. The disclosed devices and methods further make use of differential microbubble cavitation (DMC) signal processing to obtain high-quality cavitation signals for use in cavitation localization using the sensors.

In various aspects, the disclosed transcranial cavitation devices include a minimal set of four sensors for 3D transcranial localization of microbubble cavitation. The differential microbubble cavitation (DMC) signals were obtained for each sensor by subtracting signals received without and with microbubbles under the same FUS sonication condition. The DMC signals extracted the acoustic emissions from the microbubbles, thereby effectively enhancing the signal-to-noise ratio and minimizing the skull effects. The TDOAs of signals obtained from different sensors were then calculated by maximum cross-correlation (MCC). At last, the 3D cavitation location was estimated using the TDOA algorithm. This method combined differential cavitation signal detection with the TDOA localization algorithm and is also referred to herein as a differential cavitation localization (DCL) method. The accuracy of DCL with and without the human skull, and its dependency on sensor position relative to the skull, FUS pressure, and FUS cycle numbers were assessed ex vivo in a water tank. Four miniaturized ultrasound sensors were used for transcranial detection of cavitation signals emitted from microbubbles flowing through a tube when sonicated by a FUS transducer in a water tank.

In various aspects, a four-sensor network combined with a differential cavitation localization method for transcranial 3D cavitation localization is disclosed. As described in the examples below, the localization accuracy was found to be within 1.5 mm at the centers of mass. In various aspects, the four-sensor network may make use of a differential cavitation level (DCL) method that subtracts signals acquired with and without microbubbles to enhance the cavitation signal-to-noise ratio and minimize the skull effect on the localization process.

Existing cavitation localization methods typically use the delay-and-sum beam forming algorithm for cavitation location using 2D and 3D PCI. The aberration of the received signals caused by the skull has to be corrected as the absolute time of arrival is needed. In various aspects, a time delay of arrival algorithm is used to localize cavitation sources transcranially based on the relative time difference of arrival of signals detected by two different sensors in the four-sensor network. The relative time difference calculation avoids the need to perform skull aberration correction, which greatly simplifies the 3D localization algorithm. As demonstrated in the examples below, the accuracy of the disclosed transcranial cavitation localization method was robust even when the sensors were positioned at different locations around the skull.

Without being limited to any particular theory, the accuracy of the disclosed cavitation localization method depends on the acoustic pressure used to induce cavitation. As demonstrated in the examples below, as this pressure increased from 0.9 MPa to 1.3 MPa, the localization results grew to be higher and unstable, for the side lobe beam from the FUS transducer used to induce cavitation was strong enough to sonicate surrounding microbubbles to the side to the FUS focal point. In various aspects, the differential cavitation level (DCL) signals may not come from a single source, so the ambiguity of the received signals increases at high FUS pressures, leading to localization instabilities. As demonstrated in the examples below, for sonication using FUS pressure between 0.2 MPa and 0.9 MPa, a range typically used in BBB-opening applications and research, the disclosed DCL cavitation localization method can perform stable and accurate transcranial localization of cavitations.

As demonstrated in the examples below, the accuracy of the disclosed DCL cavitation localization method decreased as the FUS cycle number increased. With increasing cycle numbers, the localization accuracy decreased because higher cycle numbers corresponded to longer signal lengths. For example, the length of a 100 cycles pulse with a frequency of 500 kHz in space will be about 30 cm, which may be more than three times the length of the cavitation source to the sensors. Thus, the received signal of microbubble cavitation may experience reflection and reverberation from the obstacles such as the sensor holder, water tank, and water surface. Therefore, longer pulse cases will reduce the signal-to-noise ratio (SNR) of the received signal, which reduces the positioning accuracy. One way to make the disclosed DCL cavitation localization method capable of cavitation localizing induced by the longer pulse is to increase the distance between the sensors and the source. However, typical applications of FUS-induced cavitation methods used relatively low numbers of pulse cycles. For example, conventional histotripsy treatments typically use ultrasound cycle numbers from 3 to 10 cycles, or even as low as 1.5 cycles, and FUS-BBBD facilitated the delivery of drugs to the brain efficiently and safely using short bursts of 5 cycles.

The disclosed systems and methods for transcranial cavitation localization described herein have low computational resource requirements and low hardware costs. The data generated during localization using the methods disclosed herein are small and the corresponding calculation requirements are low, which facilitates real-time monitoring and characterization of transcranial cavitations. Due to the small number of sensors, the disclosed sensor array can be freely arranged around the skull according to actual need. In some aspects, the disclosed DCL method may be incorporated into the data analysis algorithms of a wearable therapeutic device for the brain.

In some aspects, only one cavitation source per pulse is localized using the methods disclosed herein. In other aspects, the disclosed methods are used for the localization of multiple concurrent cavitation events. In these other aspects, the time domain signal of each channel is segmented according to the position of the cavitation sources. The sources within a limited range of each detector/channel corresponding to the specific time-domain segments are calibrated, and different segments will generate sets of TDOAs based on source number, thereby locating multiple results at the same time based on time-delays.

In various aspects, the size of the cavitation source may influence the localization accuracy. Without being limited to any particular theory, the cavitation sources localized using the devices and methods disclosed herein are typically relatively low volume. Further, the TDOA-based algorithm used in the disclosed localization methods was derived to localize a point source or the distance between a sensor and source that is far greater than the wavelength of the emitted wave, such as the GPS problem. Cavitations induced using higher FUS pressures will not only lead to stronger source signals but will also induce a larger size of cavitation sources. Consequently, there is a balance between signal intensity and cavitation source size. The use of higher intensity FUS to induce cavitation will enhance localization accuracy due to the stronger signals, while the larger size of the source will degrade localization accuracy.

In various aspects, the disclosed transcranial cavitation localization method may be suitable for use in a wide variety of applications. By way of non-limiting example, 3D transcranial cavitation detection is critically needed in multiple applications, such as concussion and blast-induced traumatic brain injury caused by microcavitation formed in the brain. The capability to perform transcranial cavitation detection is critical to understand the mechanism of brain injury and to locate the injury site.

By way of other non-limiting examples, cavitation induced by focused ultrasound is a physical mechanism for several emerging techniques in brain treatments. Accurately knowing the 3D location of cavitation in real-time can improve the treatment targeting accuracy and avoid off-target tissue damage.

Additional descriptions of additional aspects of the disclosed transcranial cavitation localization methods are described in the examples below.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

### Computing Systems and Devices

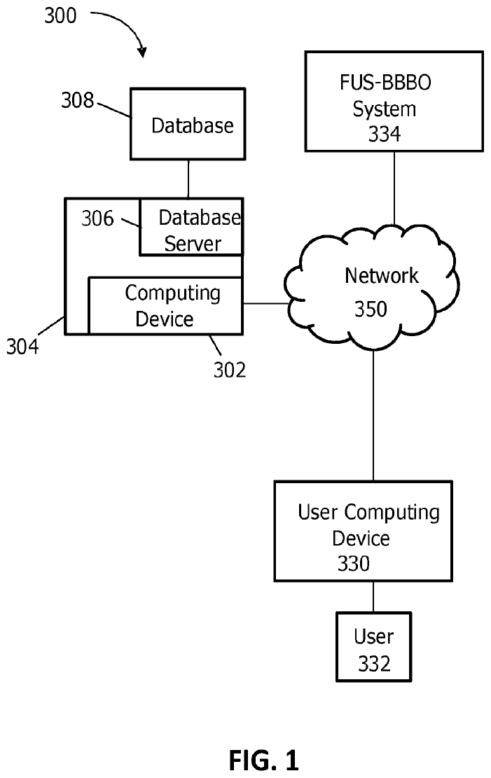

In various aspects, the disclosed FUS-BBBO and cavitation localization methods may be implemented using a computing system or computing device. FIG. 1 depicts a simplified block diagram of the system for implementing the computer-aided method described herein. As illustrated in FIG. 1, the computing device 300 may be configured to implement at least a portion of the tasks associated with the disclosed methods described herein. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330 and a FUS-BBBO system 334 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

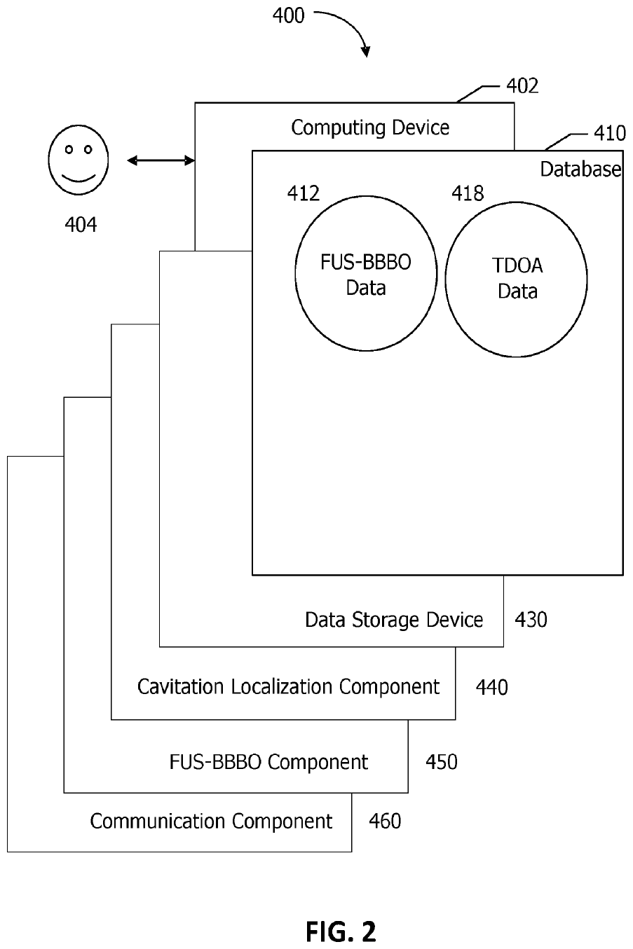

In other aspects, the computing device 302 is configured to perform a plurality of tasks associated with the disclosed computer-aided methods of performing FUS-BBBO and/or transcranial localization. In some aspects, the computing device 302, user computing device 330, and/or FUS-BBBO system 334 may be operatively connected via a network 350. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes FUS-BBBO data 412, TDOA data 418, and cavitation localization data 420. FUS-BBBO data 412 may include data used to operate a FUS-BBBO system using the individualized closed-loop feedback control of microbubble cavitation as disclosed herein. Non-limiting examples of FUS-BBBO data 412 include various measurements of cavitation signals, any parameters used to control the operation of a FUS-BBBO device, and any parameters defining equations or other algorithms used to implement the individualized closed-loop feedback control of microbubble cavitation as disclosed herein. TDOA data 418 may include data used to perform the transcranial localization of cavitation sources as disclosed herein. Non-limiting examples of TDOA data 418 include measurements of background noise and/or cavitation signals, any parameters defining equations and other algorithms used to implement the transformation of background noise and cavitation signals into differential cavitation signals as disclosed herein and/or any parameters defining equations and other algorithms used to implement localization of cavitation sources using the time difference of arrival (TDOA) method described herein.

Computing device 402 also includes a number of components that perform specific tasks. In the exemplary aspect, computing device 402 includes a data storage device 430, a cavitation localization component 440, a focused ultrasound brain-blood-barrier opening (FUS-BBBO) component 450, and a communication component 460. The cavitation localization component 440 is configured to implement transcranial cavitation localization using the determination of differential cavitation signals and/or the TDOA localization method as described herein. The focused ultrasound brain-blood-barrier opening (FUS-BBBO) component 450 is configured to implement the individualized closed-loop feedback control of microbubble cavitation as disclosed herein. The data storage device 430 is configured to store data received or generated by computing device 402, such as any of the data stored in database 410 or any outputs of processes implemented by any component of computing device 402.

The communication component 460 is configured to enable communications between computing device 402 and other devices (e.g. user computing device 330 shown in FIG. 1) over a network, such as a network 350 (shown in FIG. 1), or a plurality of network connections using predefined network protocols such as TCP/IP (Transmission Control Protocol/Internet Protocol).

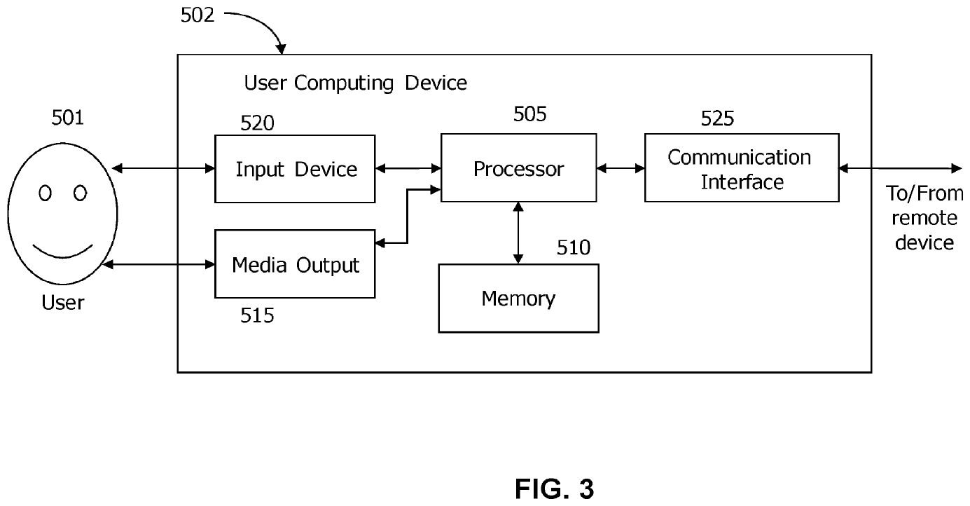

FIG. 3 depicts a configuration of a remote or user computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

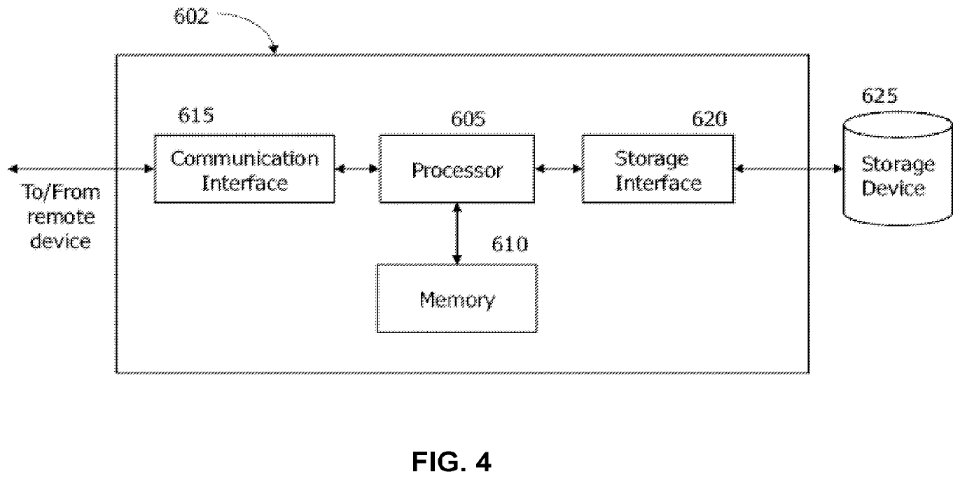

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from a user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Sensor Network Design and Experimental Setup

To develop a four-sensor network and device suitable for obtaining data suitable for use with the disclosed cavitation localization methods, the following experiments were conducted.

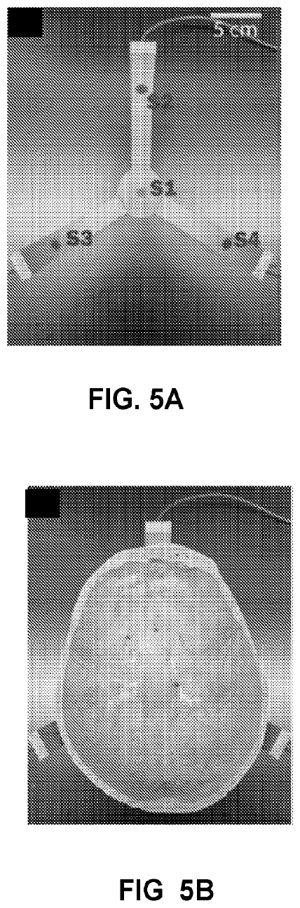

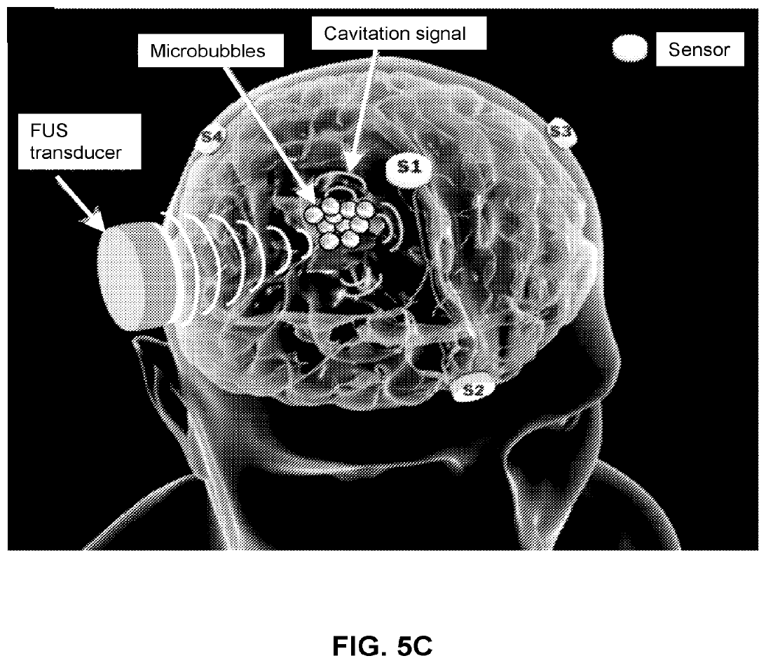

A device for 3D cavitation localization using four sensors (S1, S2, S3, and S4) was designed and fabricated. The four sensors were four identical planar ultrasound transducers. Each transducer had a center frequency of 2.25 MHz, a 6-dB bandwidth of 1.39 MHz, and an aperture of 6 mm diameter (model V323-SM, Olympus America Inc., Waltham, MA, USA). As illustrated in FIG. 5A, sensors S2, S3, and S4 were distributed equally in a horizontal plane along a circle with a radius of 78 mm. Sensor S1 was positioned along the center axis of the circle and offset vertically form the horizontal plane of sensors S2, S3, and S4 a vertical distance of 45 mm. This sensor arrangement conformed to fit with the outer contour of an ex vivo adult skullcap that was used to evaluate the developed cavitation localization method, as illustrated in FIG. 5B. The center of sensor S1 was defined as the [0, 0, 0] coordinate for the pressure measurements. FIG. 5C is a schematic diagram of the four-sensor network to locate the microbubbles sonicated by a single FUS transducer.

To reproduce experimental conditions as representative as in vivo conditions as possible the top half of a human skull was used in the experiments described herein. The skullcap, which was dry from storage in air, was immersed in water and degassed for a minimum of one week in a vacuum chamber to eliminate air bubbles trapped in the porous bones prior to conducting the experiments.

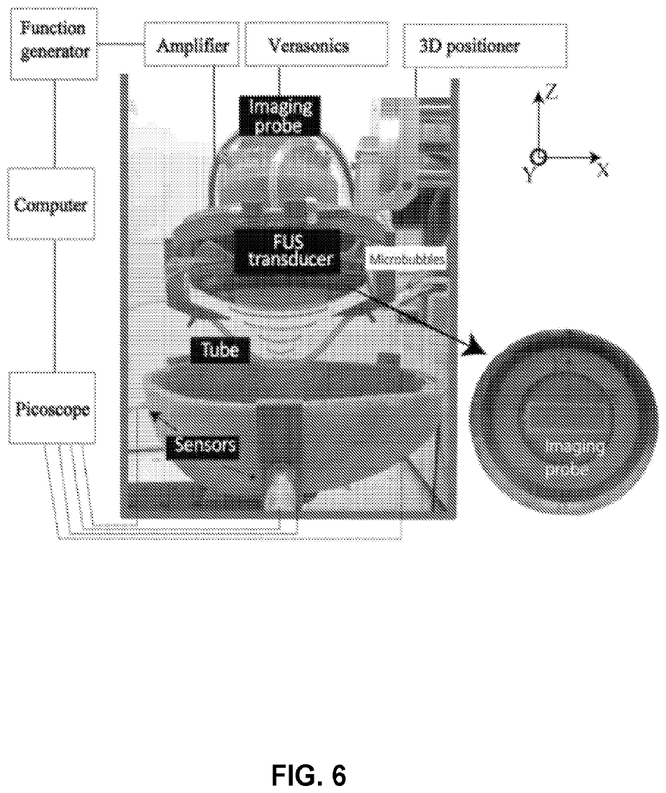

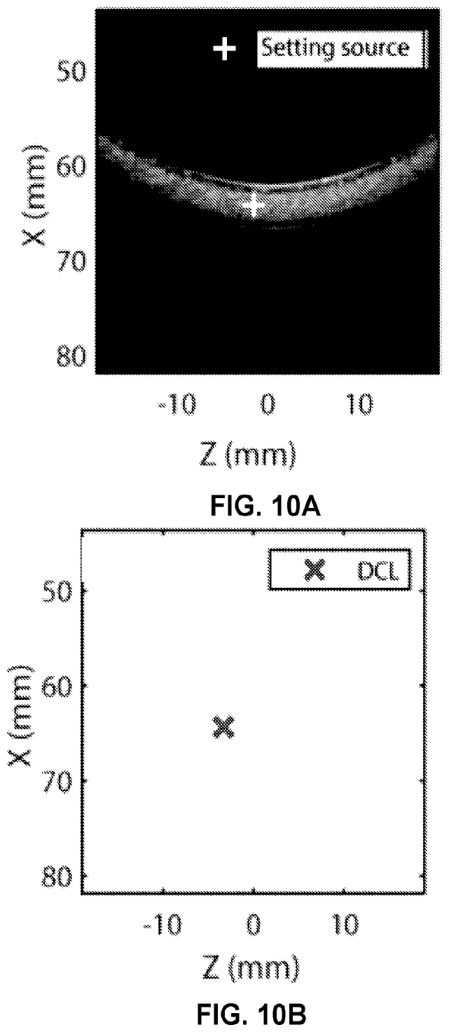

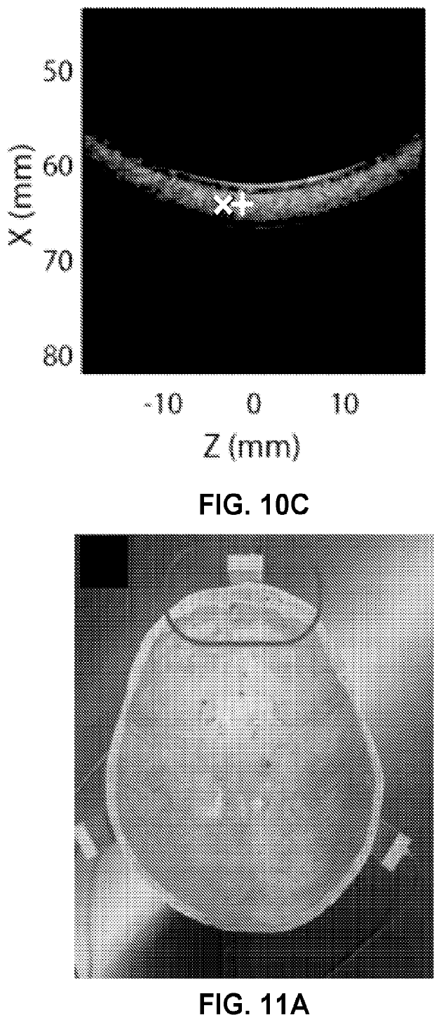

To evaluate the performance of the four-sensor network in 3D cavitation localization, a FUS transducer was used to sonicate microbubbles in a tube (3 mm inner diameter and 5 mm outer diameter) positioned above the skull. A schematic diagram of the experimental setup is provided in FIG. 6. The sensor network, skull, and tube were placed in an acrylic tank filled with room-temperature degassed water. Home-made microbubbles were diluted and injected into the tube using a syringe pump. Before dilution, the size and concentration of the microbubbles were measured using an image-based cell counter (Countess™ II FL, Thermo Fisher Scientific Inc., USA) and found to be about 6.0×105 microbubbles/mL in concentration after dilution with a mean diameter of about 4.5 μm. The FUS transducer (H204, Sonic concept, WA, USA) was driven by a function generator (Model 33500B, Keysight Technologies Inc., Englewood, CO, USA) connected to a 32-dB RF power amplifier (1020L, Electronics & Innovation, Rochester, NY, USA). The pressure at the focus of the transducer was calibrated by a hydrophone (HNC-0200, Onda, Sunnyvale, CA, USA) and all reported pressures were the measured peak negative pressures. The tube was connected with the FUS transducer using a 3D-printed frame and the FUS transducer focus was aligned at the center of the tube under the guidance of ultrasound imaging. The ultrasound imaging guidance was performed using a 128-element linear ultrasound imaging probe (ATL L7-4, Philips Healthcare) inserted into the central opening of the FUS transducer. The imaging probe was controlled by the Verasonics system (Verasonics, Inc., Redmond, WA, USA) to acquire standard B-mode images, and the location of the FUS focus was denoted by a cross overlaid on the B-mode images based on the hydrophone calibration of the FUS focus location, as illustrated in FIG. 10A.

The location of the FUS focus in reference to the center of S1, the origin of the 3D coordinates, was calibrated with the B-mode imaging. The tube was aligned with the imaging plane (XZ-plane) of the ultrasound imaging probe. To register the location of the tube with the sensor network, the position of the tube along with the FUS transducer was adjusted by a 3D motor so that the center location of S1 was aligned with the FUS focus location in the axial direction. The distance between S1 and the FUS focus was measured based on the B-mode image and the location of the FUS focus relative to the origin of the coordinates was defined. Because the positions of the S2-S4 relative to the S1 were known due to the geometry of the sensor holder, the coordinates of the center points of S2-S4 were also defined relative to the S1/origin of the coordinates.

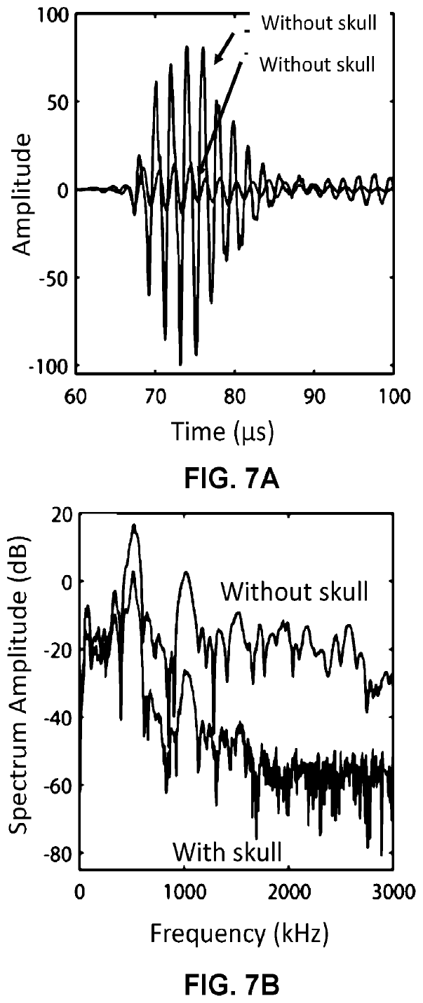

The cavitation signals detected by the four sensors were recorded with a 14-bit digital oscilloscope (Picoscope 5442D PICO Tech., UK) and stored in a computer for post-processing. FIGS. 7A and 7B illustrate the attenuation effect of the skull on the acquired signals in the time domain and the frequency domain, respectively for representative signals.

Statistical analyses were performed with GraphPad Prism (Version 8.3, La Jolla, CA, USA). Localization results of different groups were compared using unpaired t-tests (two independent groups), or a one-way analysis of variance (ANOVA) test (multiple independent groups). P-value<0.05 was used to determine statistical significance.

### Example 2—Signal Processing for Cavitation Localization

The signal processing took a two-step approach using customized software implemented using MATLAB (Mathworks, Natick, MA, USA). The DMC signals were acquired by isolating the cavitation signal from the background noise in the signals detected by the four-sensor array. The TDOA algorithm was then used to localize the cavitation source based on the DMC signals.

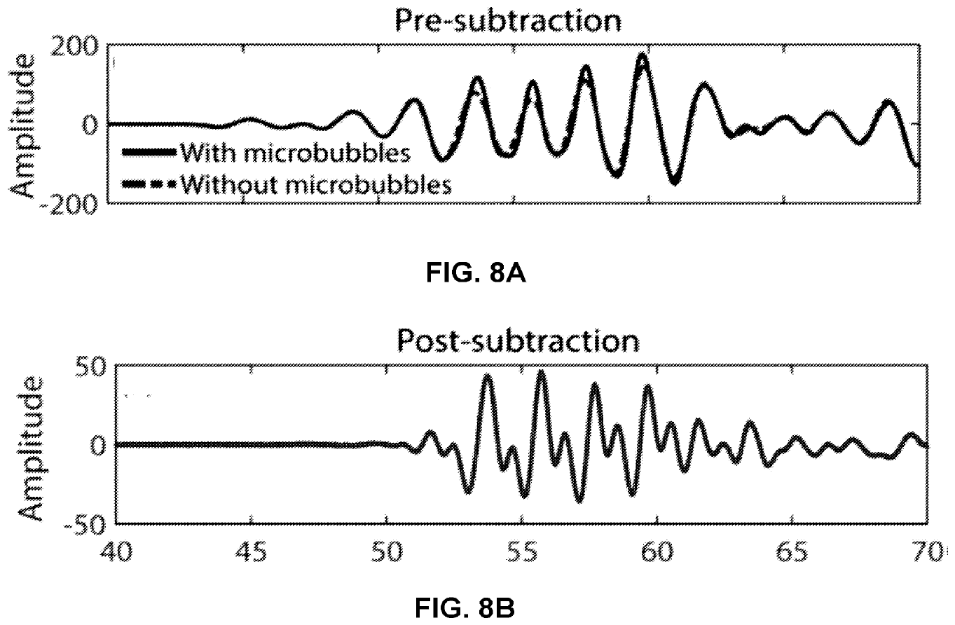

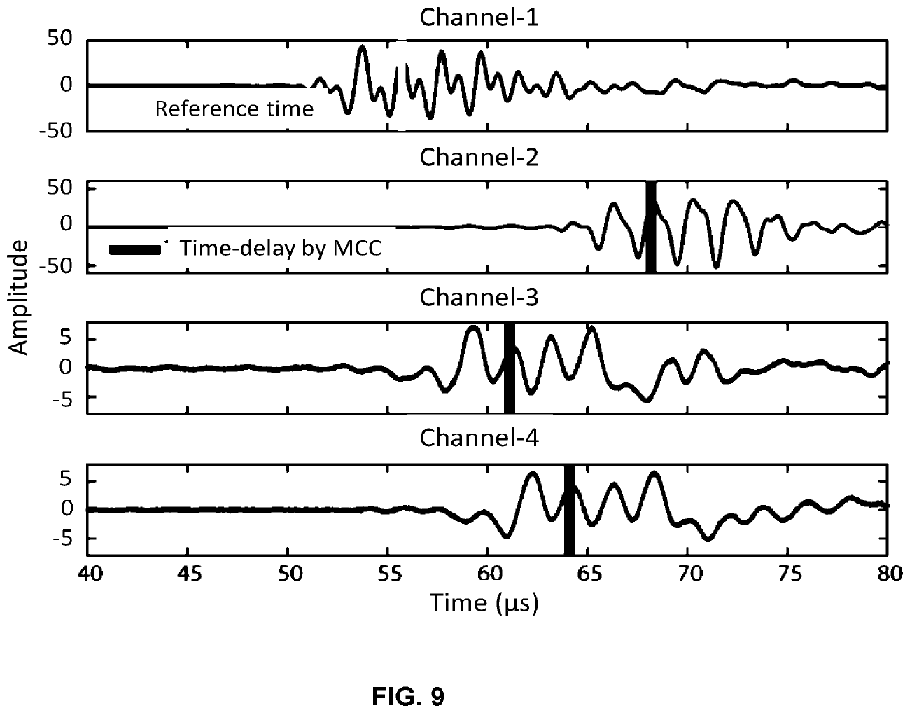

The strong attenuation of pressure signals passing through the skull results in weak harmonic components of the cavitation signal, and this attenuation increases as the frequency of the pressure signals increases. To improve the signal-to-noise ratio (SNR) of the cavitation signal, DMC signals were acquired by isolating the cavitation signal from background noise associated with scattering from the tube reflections and reverberations from the tube, holders, skull, and other intervening structures, as well as the second harmonics generated in FUS wave propagation. As illustrated in FIG. 8A, signals from FUS sonication were obtained without microbubbles for use as the reference signal. Signals from FUS sonication were then acquired with microbubbles using the same FUS setting. The reference signal (without microbubbles) was subtracted from the signals obtained with microbubbles to extract the DMC signal, which contained only the signal from microbubble cavitation, as illustrated in FIG. 8B.

Estimated TDOAs used for cavitation source location were estimated using a TDOA algorithm. The TDOA algorithm yielded three nonlinear equations with three unknowns corresponding to the source location coordinates along the x, y, and z axes. Here, let the source be at an unknown position (x, y, z), whereas, the sensors at known locations (xᵢ, {i=1,2,3,4}), so the squared distance rᵢ² between the source and sensor i is given by Eqn. (1):

$${r}_{i}^{2} = {K}_{i} - 2 {x}_{i} x - 2 {y}_{i} y - 2 {z}_{i} z + {x}^{2} + {y}^{2} + {z}^{2} , i = 1 , 2 , 3 , 4 \qquad ( 1 )$$

$$where {K}_{i} = {x}_{i}^{2} + {y}_{i}^{2} + {z}_{i}^{2} , {r}_{i}^{2} = {( {r}_{1 , i} + {r}_{1} )}^{2} , and {r}_{1 , i} = {r}_{i} - {r}_{1} .$$

r_{1,i} can be acquired based on time-difference of arrival r_{1,i}=c(t₁−tᵢ), where c is the speed of sound in water estimated using water temperature. rᵢ can be written as Eqn. (2):

$${r}_{i , 1}^{2} + 2 {r}_{i , 1} {r}_{1} = - 2 {x}_{i , 1} x - 2 {y}_{i , 1} y - 2 {z}_{i , 1} z + {K}_{i} - {K}_{1} \qquad ( 2 )$$

$$where {x}_{i , 1} = {x}_{i} - {x}_{1} , {y}_{i , 1} = {y}_{i} - {y}_{1} , and {z}_{i , 1} = {z}_{i} - {z}_{1} .$$

Eqn. (2) can be defined as a set of nonlinear equations whose solution gives (x, y, z), as expressed in Eqn. (3):

$$[ \begin{matrix}x \\ y \\ z\end{matrix} ] = - {[ \begin{matrix}{x}_{2 , 1} & {y}_{2 , 1} & {z}_{2 , 1} \\ {x}_{3 , 1} & {y}_{3 , 1} & {z}_{3 , 1} \\ {x}_{4 , 1} & {y}_{4 , 1} & {z}_{4 , 1}\end{matrix} ]}^{- 1} \times \{ [ \begin{matrix}{r}_{2 , 1} \\ {r}_{3 , 1} \\ {r}_{4 , 1}\end{matrix} ] {r}_{1} + \frac{1}{2} [ \begin{matrix}{r}_{2 , 1}^{2} - {K}_{2} + {K}_{1} \\ {r}_{3 , 1}^{2} - {K}_{3} + {K}_{1} \\ {r}_{4 , 1}^{2} - K {4}_{2} + {K}_{1}\end{matrix} ] \} \qquad ( 3 )$$

Inserting this intermediate result (x, y, z) from Eqn. (3) into Eqn. (1) at i=1 produces a quadratic equation in r₁. Substitution of the positive root of r₁ back into Eqn. (3) produces the solution. On some occasions, there may be two positive roots that produce two different answers. The solution ambiguity can be resolved by restricting the transmitter to lie within the region of interest. Linearizing r_{i,1} by Taylor-series expansion and then solving iteratively is another method of obtaining a solution, but this method may increase the computational complexity and the calculation may not converge to a solution.

### Example 3—Effect of Skull on Accuracy of DCL Cavitation Localization

To assess the accuracy of cavitation locations determined using the DCL method disclosed herein, the following experiments were conducted.

The location accuracy of the disclosed DCL algorithm was calculated by the offset of the locations estimated using the DCL method in 3D relative to the FUS focus location determined as described in Example 1. FIG. 10A shows the setting position of the cavitation source, which served as ground truth to validate the DCL localization results. FIG. 10B shows the localization results obtained using the DCL method on X-axis (Width) and Z-axis (Depth); the value of the Y-axis is perpendicular to the display plane. FIG. 10C combined the results shown in FIGS. 10A and 10B to evaluate the accuracy of the DCL method.

Four sets of localization experiments were carried out in order to assess the accuracy of the DCL method, and study the effects of the key parameters on its performance including with and without skull effects, sensor position, FUS pressure, and pulse cycle number.

To assess the effect of the skull on the accuracy of the 3D cavitation localization, the FUS transducer and microbubble tube were mounted to the three-axis positioner and moved in 10 mm increments for a total of 60 mm along the x-axis, y-axis, and z-axis, respectively. At each location, the FUS transducer was excited by a 5-cycle pulse with a center frequency of 500 kHz at a 1-Hz pulse repetition frequency (PRF) to transmit a focused beam into the microbubble tube; the in situ peak negative pressure at the FUS focus point was 0.4 MPa. Cavitation source tracking was performed over 19 locations, and each location was measured 30 times. All measurements were divided into three independent experiments conducted over three days to ensure the reliability of the acquired data. Repeated experiments were performed with and without the skull to evaluate the effects of the skull on the accuracy of the 3D cavitation localization using the disclosed DCL method. Cavitation localization with the skull was conducted with the four-sensor network position as shown in FIG. 10A.

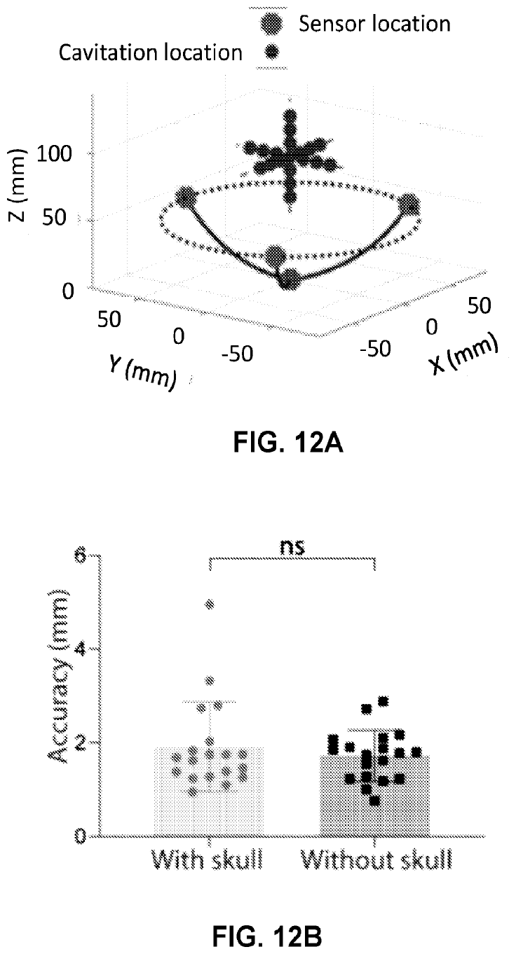

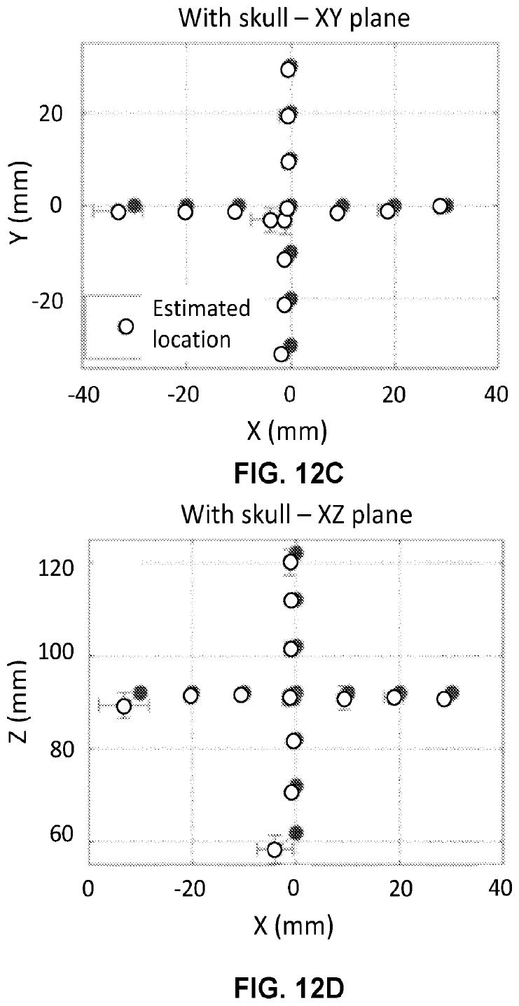

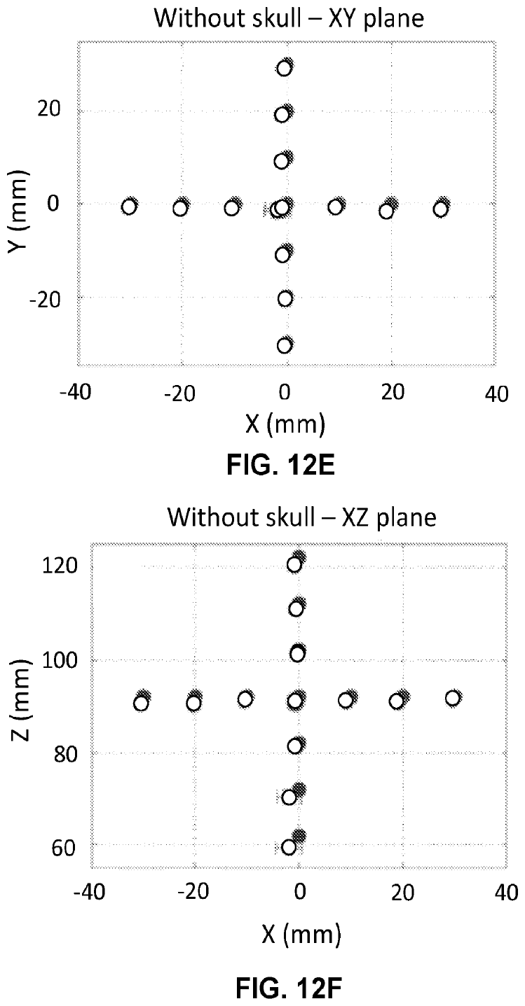

The positions of sensors and sources were graphed within a 3-D coordinate system as shown in FIG. 12A. A total of 19 sources were distributed evenly within a 60 mm×60 mm×60 mm cubic space. The accuracy of the DCL method without skull and with the skull was computed as described above and the results are presented in the bar plot of FIG. 12B. FIGS. 12C and 12D summarize the tracking accuracy of the transcranial cavitation source in the XY and XZ planes, respectively; each lighter circle represents the mean value over 10 replicates and the error bars represent the deviation along the horizontal and vertical directions, respectively. The tracking accuracy without intervening skull tissue is summarized in the XY plane (FIG. 12E) and the XZ plane (FIG. 12F), respectively. The average accuracy was 1.91±0.96 mm with the skull present and 1.73±0.54 mm without the intervening skull tissue. No statistically significant effect of skull tissue on localization accuracy based on the mean values of localization results was exhibited.

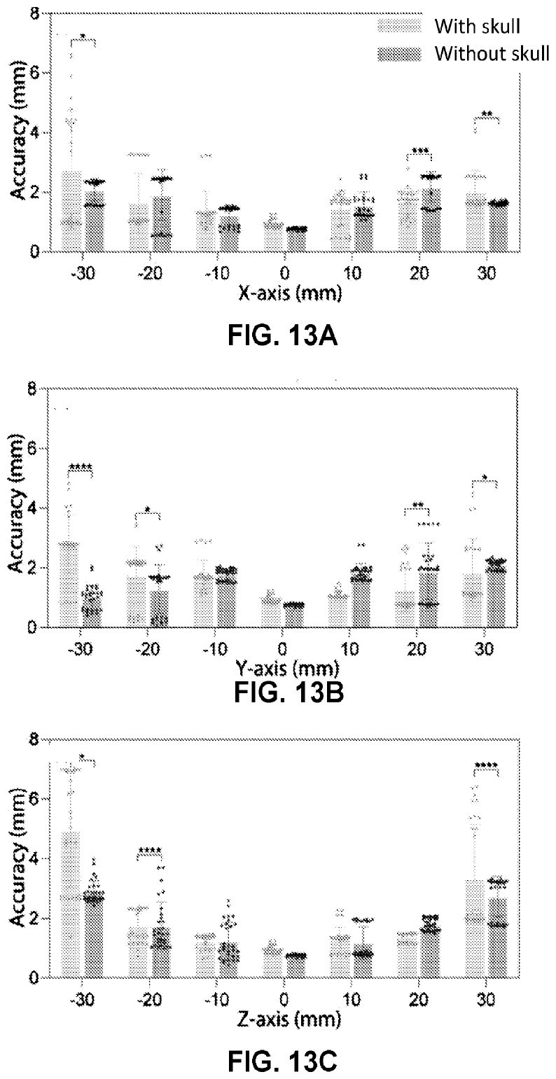

FIGS. 13A, 13B, and 13C summarize the effects of the skull on localization accuracy for specific cavitation source locations. FIGS. 13A, 13B, and 13C are derived from the results summarized in FIGS. 12C, 12D, 12E, and 12F by plotting mean and standard deviations of cavitation source localizations with and without skull as a function of locations along the X-, Y-, and Z-axis, respectively. When the cavitation source is within 10 mm of the geometric center of the four-sensor network, the presence of the skull does not significantly impact the positioning results. However, for positions x=±30, y=±30, and z=±30, the accuracy without intervening skull tissue is significantly higher than the corresponding localizations obtained through the skull. As the cavitation source gradually moved away from the geometric center of the four-sensor network, the presence of the skull increasingly reduced the localization accuracy.

The results of these experiments found no statistically significant effect of skull tissue on localization accuracy based on the mean values of localization results of the cavitation sources described above.

### Example 3—Effect of Sensor Location on Accuracy of DCL Cavitation Localization

To assess the effect of sensor placement on the accuracy of cavitation localizations performed using the disclosed DCL method, the following experiments were conducted.

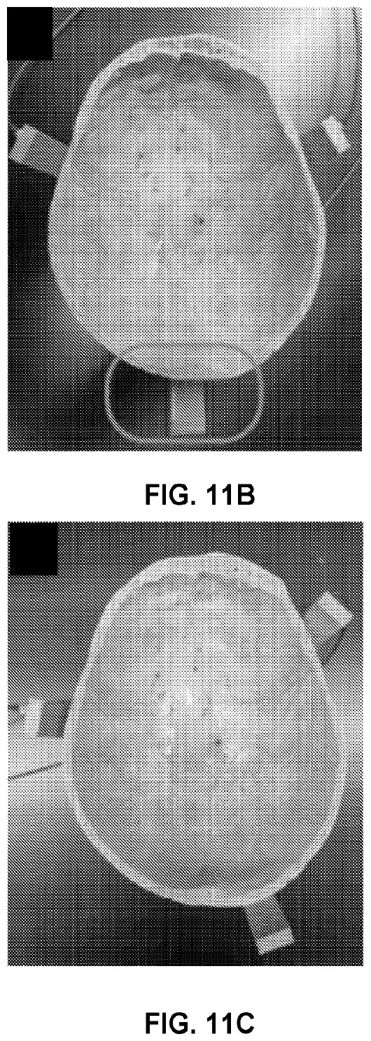

Landmark structures on the skull (occipital crest and frontal crest) were used as references to select the positioning of the sensors. The occipital and frontal crests are thicker than other regions of the skull and the internal microstructures of these two regions are more complex than within other regions of the skull. Three representative positions for the sensors were selected in this study with S2 positioned at the occipital crest (FIG. 11A), frontal crest (FIG. 11B), and off the crests (FIG. 11C). The cavitation source was set to the geometric center of the sensor network and the FUS parameters used in these experiments were matched to the parameters described in Example 2.

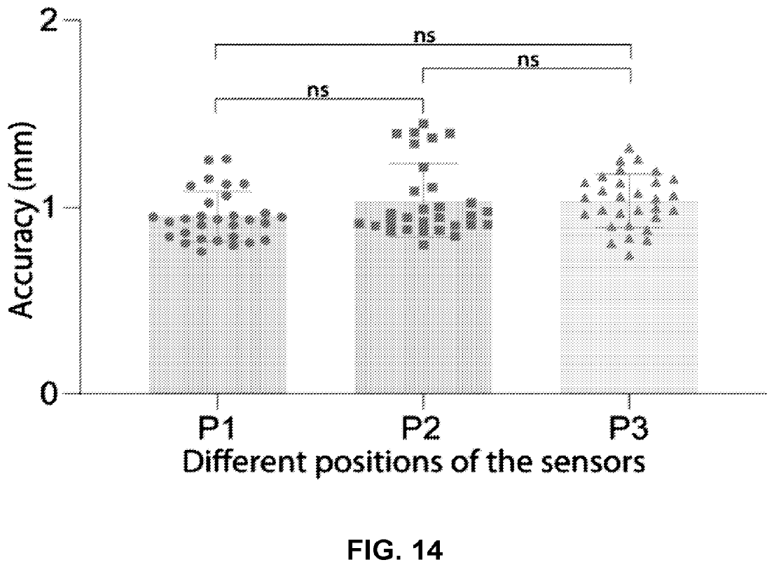

FIG. 14 summarizes the accuracy of the transcranial cavitation source localizations obtained using the disclosed DCL method for three different orientations of the sensors of the four-sensor network relative to the skull. The accuracy for the case of the occipital crest (P1), frontal crest (P2), and off the crest (P3) was 0.95±0.13 mm, 1.04±0.20 mm, and 1.03±0.14 mm, respectively. Based on the tests of statistical analysis, the contact position of the sensors with the skull had no significant effect on localization accuracy.

### Example 4—Effect of FUS Pressure on Accuracy of DCL Cavitation Localization

To assess the effect of FUS pressure on the accuracy of cavitation localizations performed using the disclosed DCL method, the following experiments were conducted.

The cavitation source was set to the geometric center of the sensor network as described in Example 3, the FUS was maintained at 5 cycles, and the four-sensor network was positioned on the skull as shown in FIG. 11A. FUS peak negative pressures ranging from about 0.2 MPa to about 1.3 MPa were used to sonicate microbubbles inside the microbubble tube phantom to assess pressure amplitude effects on DCL localization performance.

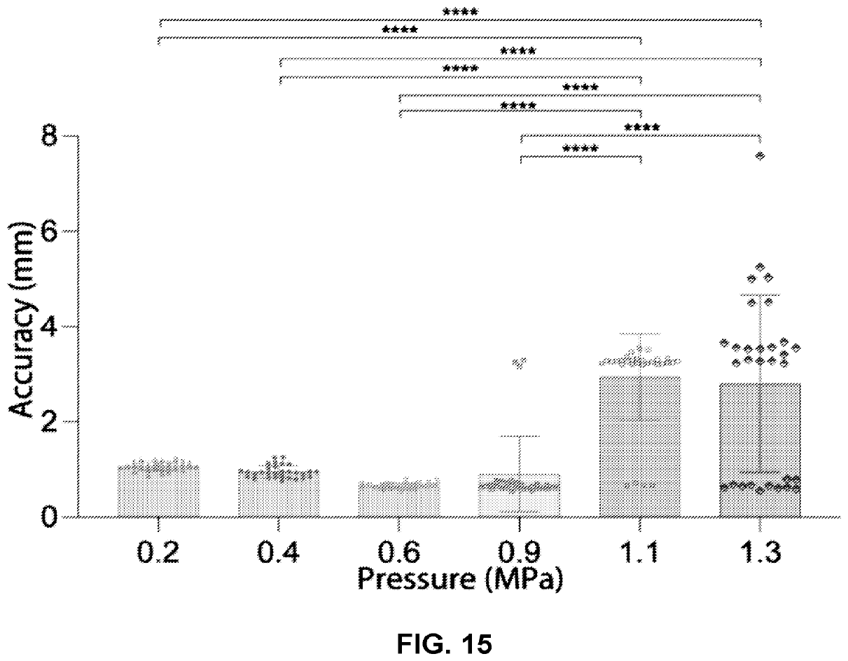

FIG. 15 summarizes the accuracy of the transcranial cavitation source localizations obtained using the disclosed DCL method as a function of FUS pressure. The FUS pressures used in these experiments were 0.2, 0.4, 0.6, 0.9, 1.1, 1.3 MPa, and the corresponding localization accuracies were 1.06±0.09 mm, 0.95±0.13 mm, 0.65±0.06 mm, 0.91±0.79 mm, 2.94±0.91 mm and 2.80±1.86 mm, respectively. When the FUS peak negative pressure was increased from 0.2 to 0.6 MPa, the mean value of accuracy decreased slightly. For FUS peak negative pressures ranging from 0.9 MPa to 1.3 MPa, the localization results increased and grew increasingly unstable. Based on a statistical analysis of the above results, the localization accuracy at FUS pressures ranging from 0.2 MPa to 0.9 MPa was significantly different from the corresponding results for FUS pressures ranging from 1.1 MPa to 1.3 MPa.

### Example 5—Effect of FUS Pressure Cycles on Accuracy of DCL Cavitation Localization

To assess the effect of the FUS pulse cycle number on the accuracy of cavitation localizations performed using the disclosed DCL method, the following experiments were conducted.

For these experiments, the cavitation source was fixed at the center of the four-sensor network, the FUS peak negative pressure was maintained at 0.4 MPa, and the four-sensor network was positioned in the skull as shown in FIG. 11A. Different cycle lengths of the FUS source ranging from 5 cycles to 1000 cycles were tested to sonicate microbubbles to determine the optimal signal length.

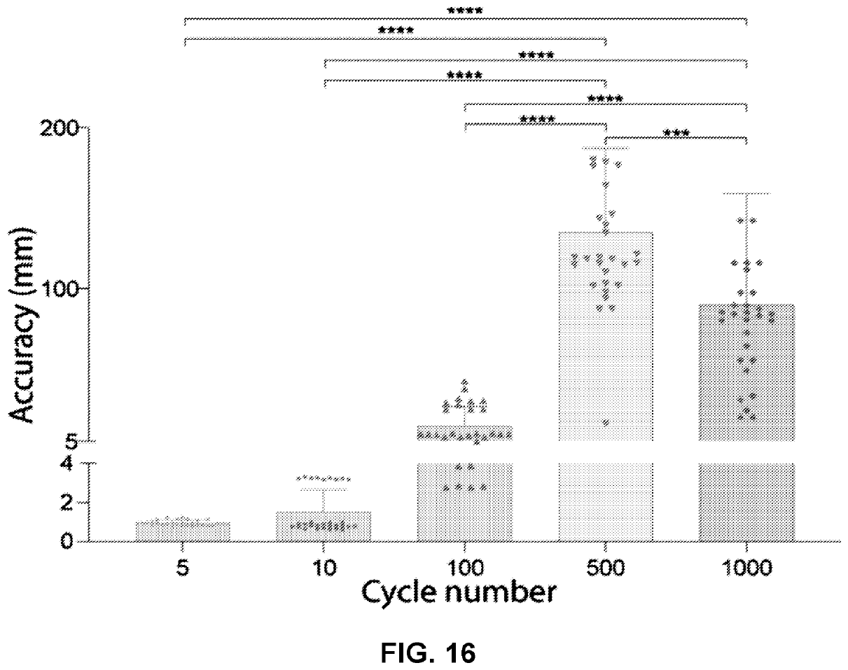

FIG. 16 summarizes the dependence of the accuracy of the transcranial localization on the number of FUS cycles. The numbers of FUS cycles tested in these experiments were 5, 10, 100, 500, and 1000 cycles, and the corresponding localization accuracies were 0.95±0.13 mm, 1.52±1.14 mm, 14.61±11.96 mm, 135.2±52.07 mm, and 90.17±68.99 mm, respectively. As the number of cycles increased, the positioning accuracy of the DCL method decreased and became increasingly unstable. Statistical analysis of the above results indicated that the localization accuracy at FUS cycle numbers 5 and 100 was significantly different from the corresponding localization accuracy at FUS cycle numbers 500 and 1000. In addition, the localization accuracies at FUS cycle numbers of 500 and 1000 were significantly different from each other.

### Example 6—Effect of FUS Pressure Cycles on Accuracy of DCL Cavitation Localization

To investigate the feasibility of using a four-sensor network for transcranial 3D localization of microbubble cavitation, the following experiments were conducted.

Cavitation is the dominant physical mechanism for focused ultrasound (FUS)-activated cavitation-mediated therapies in the brain. Accurately knowing the 3D location of cavitation in real-time can beneficially improve the treatment targeting accuracy and avoid off-target tissue damage. However, the skull induces strong phase and amplitude aberrations to the cavitation signals and presents significant challenges to the localization of transcranial cavitations. Existing techniques for 3D cavitation localization use hemispherical multielement arrays combined with passive beamforming and adaptive skull-specific correction algorithm. However, these techniques require expensive equipment and time-consuming computational methods that limit the application of existing methods in real-time cavitation localization, which is urgently needed to ensure the safety and efficacy of the FUS treatment.

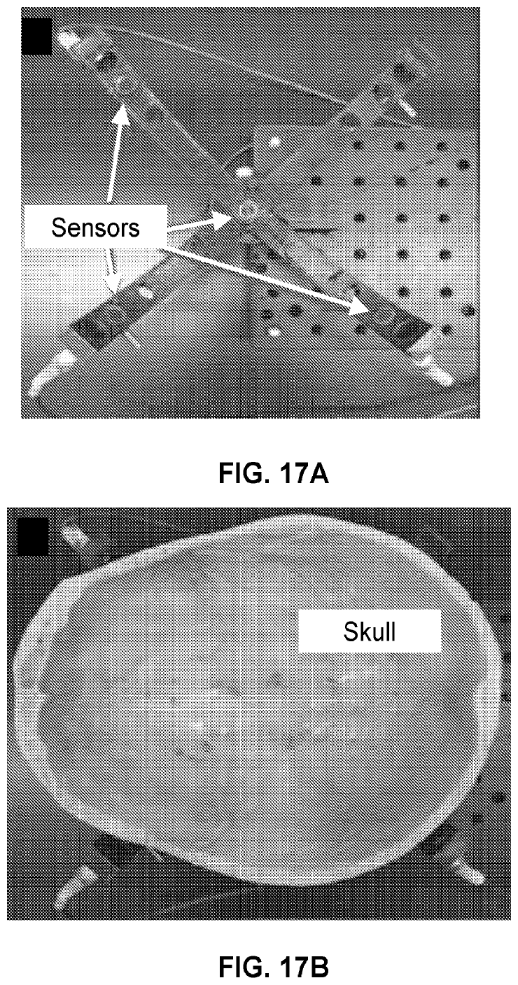

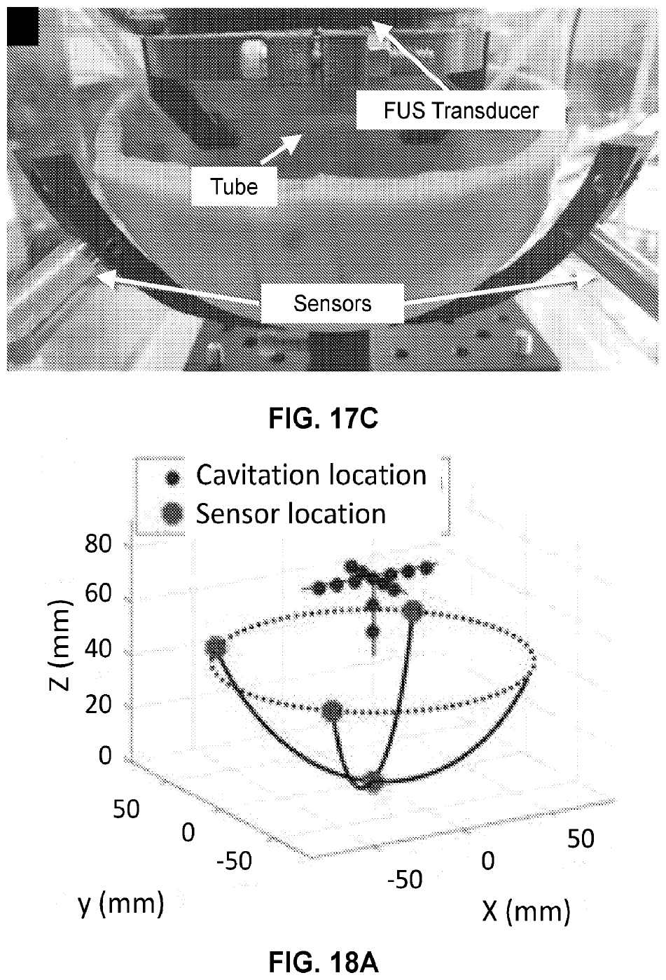

A device for 3D cavitation localization was designed and fabricated (FIG. 17A). The device consisted of four sensors (Olympus V323-SM) distributed on a hemisphere with a diameter of 18 cm. The performance of the device was evaluated using an ex vivo human skull setup (FIG. 17B). In this setup, microbubbles (˜1×10⁶ microbubbles/mL) were injected into a tube phantom (3 mm inner diameter and 5 mm outer diameter) positioned inside the skull cavity (FIG. 17C). The microbubbles were activated by a FUS transducer (1 Hz pulse repetition frequency, 75 cycles in pulse length, and 1.5 MHz driving frequency) at an estimated in situ peak negative pressure of 6.5 MPa. The FUS transducer and the tube were moved by a 3D stage to different locations within the skull setup. The FUS transducer was coaxially aligned with the ultrasound imaging probe. B-mode images were acquired before and after FUS sonication to determine the location of the cavitation event based on image contrast changes. The acoustic emissions from the FUS-activated microbubbles were passively detected and saved for postprocessing. The signals were filtered to only keep the subharmonic frequencies (750 kHz with 300-kHz bandwidth), which were thought to contain the cavitation signals. The time delays between signals received by the four sensors were measured by detecting the maximum inter-correlation between them. The position of the cavitation source was calculated using a method similar to that usually used in the global positioning system (GPS).

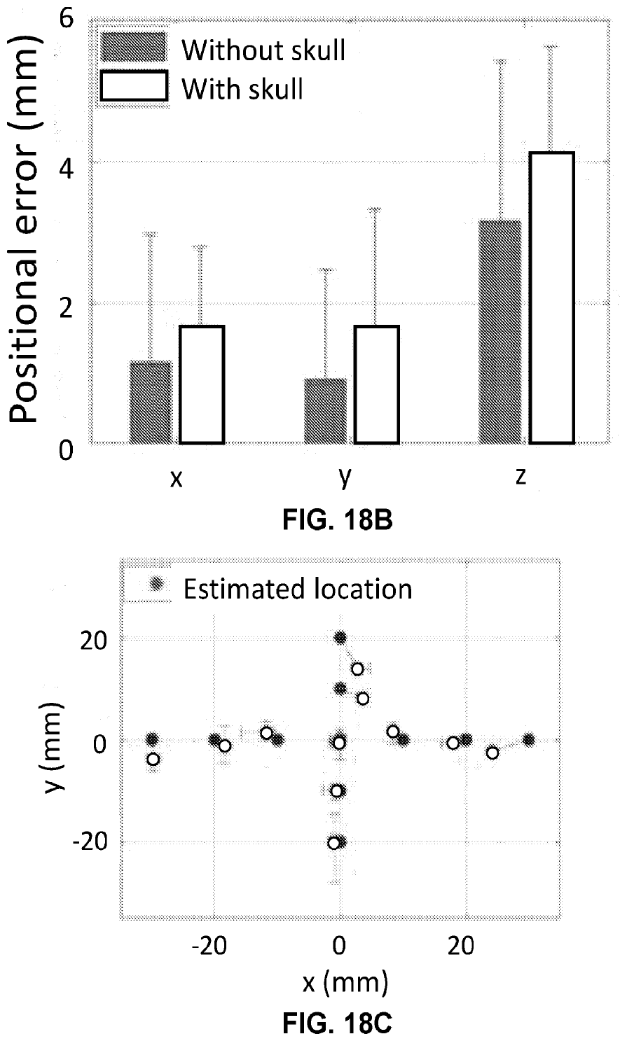

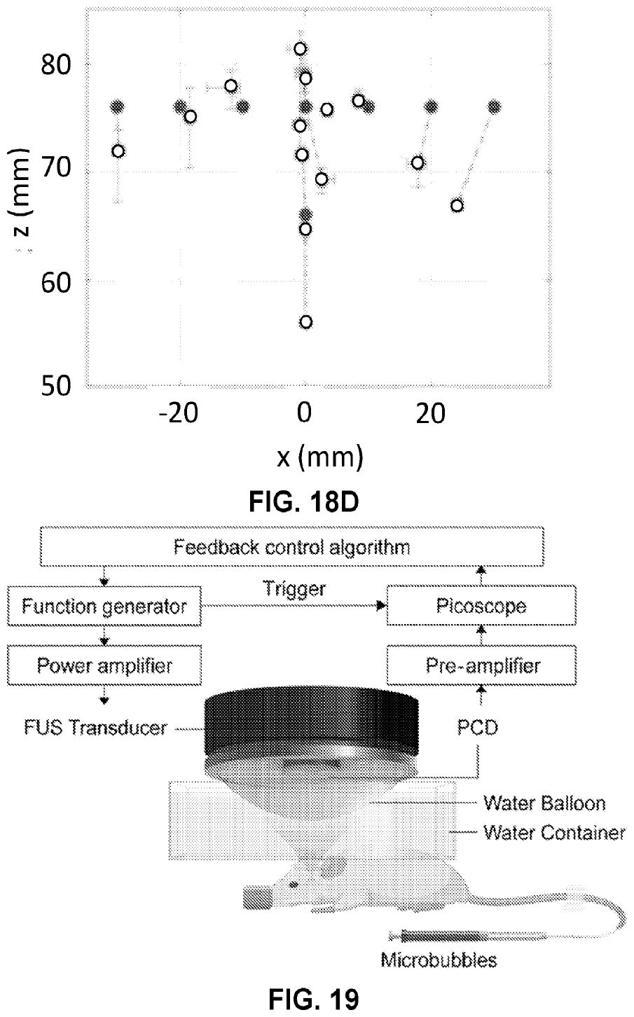

FIG. 18A shows a schematic diagram of the spatial positions of the four sensors of the 3D cavitation localization device described above, as well as the movements of the cavitation source within the experimental setup. FIG. 18B shows the positional error along the x-, y-, and z-axis determined based on cavitation signals detected with and without intervening skull; error bars denote the standard deviation of measured locations. The positional errors of transcranial cavitation localizations along the x, y, and z axes were 1.7±1.2 mm, 1.6±1.7 mm, and 4.1±1.5 mm, respectively. For comparison, the positional errors of cavitation localizations based on signals detected in the absence of the skull were 1.2±1.8 mm, 0.9±1.6 mm, and 3.1±2.3 mm, respectively. FIGS. 18C and 18D summarize the tracking accuracy of transcranial cavitation localization in the xy plane and xz plane, respectively. The lighter circle markers represent the mean value over four replicates and the bars represent the upper and lower standard deviation along the horizontal and vertical directions. Higher accuracy was achieved along the x and y axes as compared to the z-axis. Larger deviations were observed at locations further away from the center of the detector array as compared to corresponding deviations of measurements obtained for cavitations close to the center.

The results of these experiments confirmed the feasibility of using a four-sensor network for transcranial cavitation localization in 3D. The disclosed method achieved mean accuracies of 1.7 mm, 1.6 mm, and 4.1 mm along the x, y, and z axes, respectively. The disclosed method determined the cavitation location in 3D with a low computation cost, making it possible for real-time cavitation localization in 3D.

### Example 7—Evaluation of Closed-Loop Feedback Control of Microbubble Cavitation for FUS-BBBO

To evaluate the safety and reliability of the closed-loop feedback control method for microbubble cavitation for focused ultrasound brain blood barrier opening (FUS-BBBO) disclosed herein, the following experiments were conducted.

FUS-BBBO using the disclosed feedback control method was used to open the BBB for delivery of Evans blue dye into the brains of mice.

### Experimental Animals

25 Swiss mice (8-10 weeks, ˜25 g body weight, female, Charles River Laboratory, Wilmington, MA, USA) were randomly assigned into five groups (n=5 for each group) to evaluate five different TCLs using the disclosed algorithm. Swiss mice (8-10 weeks, ˜25 g body weight, female, Charles River Laboratory, Wilmington, MA, USA) were housed in a room maintained at 22° C. and 55% relative humidity, with a 12-h/12-h light/dark cycle and access to standard laboratory chow and water. 25 Swiss mice were randomly assigned into five groups (n=5 for each group) to evaluate five different TCLs using the proposed algorithm. Four Swiss mice were selected to evaluate an existing closed-loop feedback control algorithm with the TCL defined based on the detection of sub-harmonics. During all experiments, mice were anesthetized with 1.5-2% isoflurane and stabilized using a stereotaxic apparatus (Kopf, Tujunga, CA, USA). A heating pad with a temperature kept at ˜38° C. was used to maintain the mouse's body temperature. Mice were prepared for FUS sonication by removing fur on top of the head with a depilatory cream (Nair, Church & Dwight Co., NJ, USA) and coupled to a water container using ultrasound gel. A catheter was placed into the tail vein for microbubbles and Evans blue injection.

### FUS-BBBO System

The mice were subjected to FUS-BBBO using the system illustrated in FIG. 19. A single-element FUS transducer with an aperture of 75 mm, a radius of curvature of 60 mm, and a center opening of 25 mm in diameter was used to deliver FUS to the mice. The FUS transducer was impedance matched to operate at 1.5 MHz and driven by an arbitrary waveform generator (Agilent 33500B; Agilent Technologies, Loveland, CO, USA) that was connected to a 53-dB power amplifier (1020 L; E&I, Rochester, NY, USA). The FUS transducer was attached to a 3D stage to facilitate the targeting of the transducer output. The acoustic pressure fields generated by the FUS transducer were calibrated using a needle hydrophone (HNP-0200; Onda Inc., Sunnyvale, USA) in a degassed water tank. The axial and lateral full-width-at-half-maximum (FWHM) dimensions of the FUS transducer were 8.3 mm and 1.1 mm, respectively. The peak negative pressures of the FUS transducer at different voltage input levels were measured at the focus of the transducer in a water tank. A 3-D printed bar with a sharp tip was manufactured to facilitate precise targeting of a specific brain location. The tip of the bar was positioned in close proximity to the top of the hydrophone when the FUS transducer was switched to the bar for use as a pointer. The pointer was then used to indicate the FUS focus. The tip of the pointer was moved by the 3D stage to be aligned with the lambda on the mouse skull, which was visible through the mouse's skin. The pointer was then switched to the FUS transducer. The transducer was moved 1 mm lateral and 1 mm posterior, and 4 mm ventral to target the brainstem, which was selected to represent a targeted brain location. A single-element ultrasound transducer (I5P10, Guangzhou, China) with a center frequency of 4.7 MHz and a 6-dB bandwidth of ±1.9 MHz was inserted through the center hole of the FUS transducer and both transducers were maintained in confocal alignment using a 3-D printed housing. A single-element ultrasound transducer was used to acquire cavitation emissions from the microbubbles during FUS sonication as for passive cavitation detection (PCD). This PSD transducer was connected to a 22 dB pre-amplifier and a PicoScope (5244B, Pico Technology, Cambridgeshire, UK). The PicoScope was triggered by the arbitrary waveform generator to synchronize PCD data acquisition with the FUS sonication. The signal acquired by the PCD was sampled at 40 MHz. All the equipment was controlled by the PC using a custom MATLAB program.

### FUS-BBBO Under Real-Time Closed-Loop Feedback Control

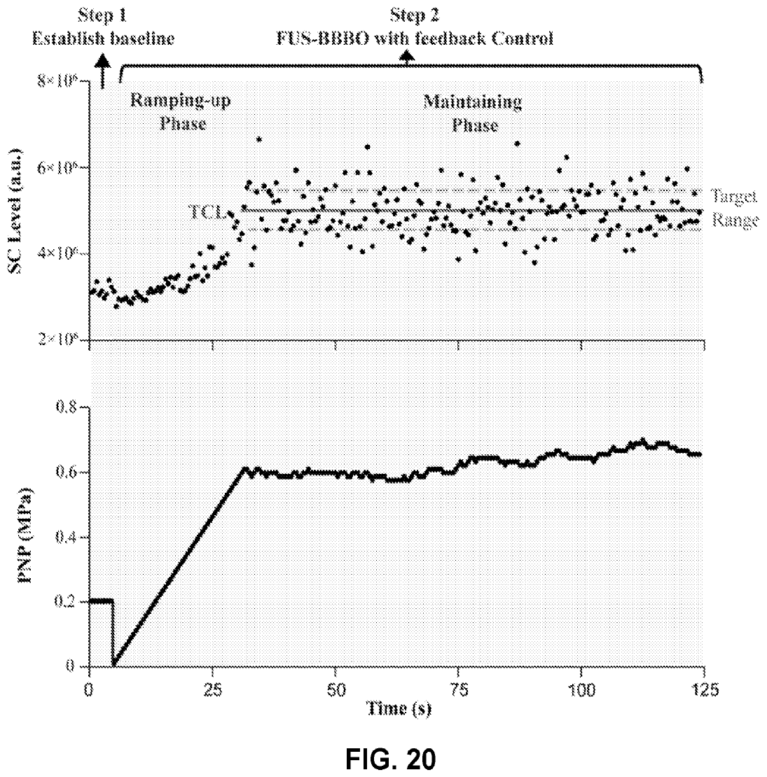

A microbubble contrast agent (Definity, Lantheus Medical Imaging, North Billerica, MA) was diluted using sterile saline to a final concentration of approximately 8×10⁸ microbubbles per mL. A bolus of diluted microbubble contrast agent (volume=30 μL) was injected intravenously into each mouse through a tail vein catheter. The injection was performed using a computer-controlled syringe pump (NE-1600; New Era Pump Systems Inc.). Microbubbles infusion was started 15 s before FUS sonication to allow microbubbles to flow through the tail vein catheter and reach the mouse brain. The infusion lasted until the end of sonication at a constant rate of 12.8 μL/min. All mice were treated by FUS with output pressure controlled in real-time using the disclosed PCD-based closed-loop feedback control algorithm. The treatment procedure followed a two-step process. A representative example of sonication sequence and detected cavitation level is illustrated in FIG. 20.

A baseline stable cavitation (SC) level was established for each mouse with dummy FUS sonication after injecting the microbubbles. FUS sonication was performed using a pulse repetition frequency of 2 Hz, a pulse length of 6.7 ms burst (i.e., duty cycle: 1.33%), and a sonication duration of 5 s. The output pressure of FUS was 0.2 MPa (all pressures reported were the peak negative pressures calibrated in water). This pressure was selected because it was the lowest pressure at which the microbubble cavitation signal was higher than the noise level without microbubble injection and lower than the pressures needed to induce BBB disruption. During the sonication by each FUS pulse, acoustic emission from microbubbles was recorded by the PCD transducer and processed by a Fast-Fourier transform (FFT) algorithm. SC level was calculated by summing the magnitude of the spectrum within a ±0.02 MHz bandwidth at the third harmonic (i.e., 4.5 MHz) of the FUS transducer. The third harmonic emission was chosen because it was at the center frequency of the PCD transducer. Ten PCD signals were acquired, and the average of SC levels calculated from these ten signals was used to define the baseline SC level.

After establishing the baseline SC level as described above, the mice were further subjected to FUS sonication using the FUS-BBBO with real-time feedback control disclosed herein. During FUS sonication with microbubbles infusion, cavitation was monitored by PCD in real-time, and a custom closed-loop feedback control algorithm was used to control the SC level to be at different TCLs defined to be 0.5 dB, 1 dB, 2 dB, 3 dB, or 4 dB above the baseline SC level. The feedback control algorithm in these experiments consisted of a ramping-up sonication phase followed by a maintaining sonication phase, shown illustrated in FIG. 20. The ramping-up phase started at 0 MPa and increased pulse by pulse with a step of 0.013 MPa until the SC level reached the TCL. Once the SC level reached the TCL, the control algorithm switched to the maintaining phase with the acoustic pressure continuously adjusted to maintain the SC level within the target range (i.e., TCL±tolerance range) until the end of the sonication. The tolerance range was set to ±0.4 dB to reduce the sensitivity to noise. If the SC level was located within the range of TCL±tolerance range, the FUS output pressure was kept the same. For the case that SC level was higher or lower than TCL±tolerance range, the FUS output pressure of the next pulse was decreased or increased by the step size (0.013 MPa) immediately. The step size (0.013 MPa) was the minimum step size of the arbitrary waveform generator and was set for achieving fine adjustment.

### Stability of FUS-BBBO Feedback Control Method

The stability of the feedback control algorithm was determined by the good burst rate, which was calculated by the percentage of all measured SC levels in the maintaining phase that fell within the TCL±tolerance range. Higher stability represented more effective controllability among the cavitation activities. IC level was also quantified based on the acquired cavitation signals to serve as a safety check. IC level was calculated by summing the magnitude of the spectrum within a ±0.02 MHz bandwidth at 3.3 MHz. These frequencies were chosen to quantify the level of the broadband signals by avoiding harmonics and ultra-harmonics. The presence of an IC event was defined when the IC level was over 1 dB above the baseline IC level quantified based on the signals acquired during dummy FUS sonication after injecting microbubbles. Inertial cavitation (IC) probability was calculated as the percentage of IC events that were present during the maintaining phase. Higher IC probability indicated a higher occurrence of IC events and a higher potential for tissue damage.

The GraphPad Prism (Version 9.0, La Jolla, CA, USA) was used to analyze data. Differences between the two groups were determined using an unpaired two-tailed Student's t-test. A p-value<0.05 was used to determine statistical significance.

FIG. 21 shows the baseline SC levels measured for each mouse in the five groups with dummy sonication. As expected, variations in the baseline SC level were observed among different subjects. The feedback control algorithm described above maintained the SC level at TCLs of 0.5 dB, 1 dB, 2 dB, 3 dB, and 4 dB above the baseline SC level for the individual mouse; the targeted absolute SC level was different for each mouse, as illustrated in the lower right graph of FIG. 21.

The measured SC levels for each mouse in each group throughout the FUS-BBBO procedure are shown in the graphs of FIG. 22A. The plot of the mean TCL for all groups in FIG. 22A (lower right graph) illustrates the successful control of the FUS sonication to maintain the SC at different levels using the feedback control algorithm disclosed herein. As summarized in FIG. 22B, the disclosed feedback control algorithm achieved average stabilities of 78.6%, 74.0%, 65.9%, 58.2%, and 62.6% for TCLs of 0.5 dB, 1 dB, 2 dB, 3 dB, and 4 dB, respectively. FIG. 22C summarizes the measured mean IC levels for each group. Average IC probabilities were 0%, 0%, 0%, 4.5%, and 37.0% for TCLs of 0.5 dB, 1 dB, 2 dB, 3 dB, and 4 dB, respectively, as shown in FIG. 22D. IC probabilities were significantly higher at 3 dB and 4 dB relative to other groups.

### Evaluation of Drug Delivery Through BBB

Evans Blue, a widely used agent to evaluate BBB permeability changes, was used to evaluate the effectiveness of drug delivery to the brain using FUS-BBBO with the disclosed feedback control algorithm. Mice were intravenously injected with 30 μL of 4% Evans Blue immediately after FUS sonication as described above. Mice were sacrificed and perfused 30 minutes after sonication. Mouse brains were then harvested and fixed using 4% paraformaldehyde. The extracted whole brains were sectioned into 1 mm thick slices in the horizontal plane and examined by the Licor Pearl small animal imaging system (LI-COR Biosciences, Lincoln, NE) with acquisition using the 700 nm channel for imaging Evans Blue. The exposure time for fluorescence imaging was kept the same for imaging all the brain slices. The fluorescence intensity of the brains was then quantified using LI-COR Image Studio Lite software. Regions of interest (ROIs) were selected to cover the target brainstem region, and quantifications on all slices were normalized to the background ROI (i.e., background signal of tissue). For each mouse, the normalized fluorescence intensity was used to quantify the effectiveness of Evans Blue delivery concentration at the target region (i.e., ROI) as an indication of FUS-BBBD drug delivery efficiency using the disclosed feedback control method. The spatial diffusion of the Evans blue was quantified to represent the FUS-BBBO drug delivery area. Fluorescence intensities higher than 10 dB above the background signal of brain tissue were extracted as drug delivery areas and quantifications of these areas were calculated by a customized MATLAB program.

FIG. 23A shows photographs of representative brain slices and corresponding fluorescence images at each TCL (i.e. 0.5 dB, 1 dB, 2 dB, 3 dB, and 4 dB). Relative to the 0.5 dB group, the fluorescence intensities of the delivered Evans Blue increased by an average of 2.4-fold, 5.3-fold, and 8.2-fold at 1 dB, 2 dB, and 3 dB, respectively, as summarized in FIG. 23B. Significant differences in the fluorescence intensities were observed among different groups (0.5 dB versus 1 dB, P=0.0046; 1 dB versus 2 dB, P=0.0002; 2 dB versus 3 dB, P=0.0178). As summarized in FIG. 23C, drug delivery areas increased from 9.6 mm² to 112.8 mm² (11.7-fold higher) on average as the TCL was raised from 0.5 dB to 3 dB. There was a significant difference of delivery area between each TCL group (0.5 dB versus 1 dB, P=0.0313; 1 dB versus 2 dB, P=0.0036; 2 dB versus 3 dB, P=0.0001). When TCL increased to 4 dB, the fluorescence intensity and drug delivery area slightly decreased with respect to the corresponding values observed at a TCL of 3 dB.

### Safety Evaluation by Histologic Analysis

Histologic examination was performed on all mice using hematoxylin and eosin (H&E) staining. Specifically, after fluorescence imaging, the brain slices containing the targeted brainstem were fixed in 4% paraformaldehyde overnight, followed by cryoprotected with sucrose. The brain slices were sectioned horizontally into 10 μm sections and stained with H&E. Digital images of tissue sections were obtained using an all-in-one microscope (BZ-X810, Keyence, Osaka, Japan). The hemorrhage area of the stained region was extracted based on pixel hue by the built-in software of BZ-X810. The total area of red blood cell extravasation was calculated by summing all the identified pixels in the FUS-targeted side of the brainstem. The contralateral brain area without FUS sonication was used as the control.

FIG. 24A shows the representative H&E staining images of the brainstem at the level where the FUS focus was targeted. FUS was targeted at the right side of the brainstem, and the contralateral side was used as the control. As shown by the lower and higher magnification images, no hemorrhage was observed in the 0.5 dB, 1 dB, and 2 dB cases. Mild tissue damage was found in the 3 dB case, and relatively severe tissue damage was found in the 4 dB case. As summarized in FIG. 24B, group analysis found no significant differences between the FUS treated side and contralateral non-treated side in 0.5 dB, 1 dB, and 2 dB groups, but significant tissue damage was observed within the FUS-treated regions in the 3 dB and 4 dB groups.

Comparison with Existing Feedback Control Algorithm

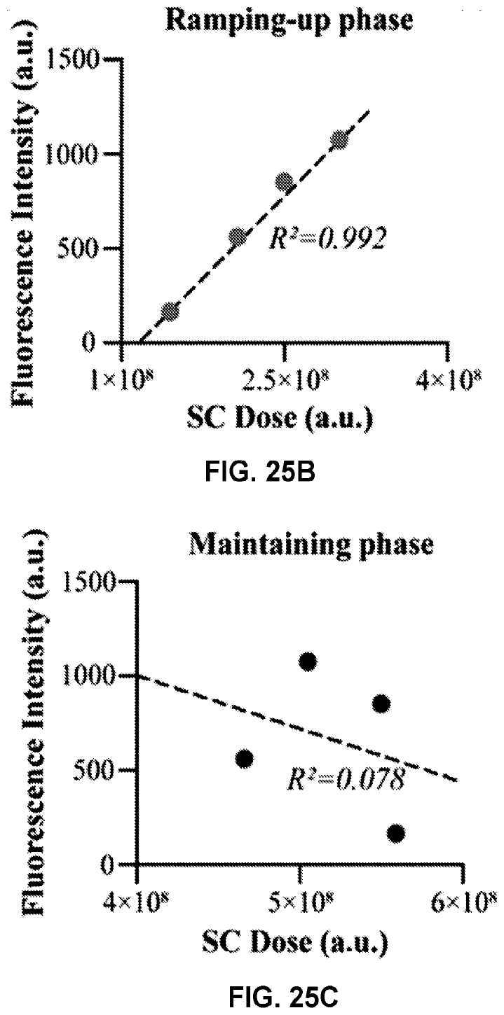

The disclosed closed-loop feedback control was evaluated relative to an existing closed-loop feedback control approach in which TCL was defined based on the detection of sub-harmonic signals. As illustrated in FIG. 25A, a pressure ramping-up was applied until the SC level reached the threshold at which subharmonic signals (i.e., emissions at 0.5 f₀, where f₀ is the fundamental frequency of the driven frequency) were detected. The acoustic pressure was then controlled throughout the rest of the procedure to maintain the SC level at 50% of the threshold. A total of 4 mice were used to test this existing approach following the same procedure described above and the mouse brains were processed in the same way for the quantification of the Evans blue delivery outcome described above.

FIG. 25A shows an example of the recorded SC level of the closed-loop feedback control algorithm with the TCL defined based on the detection of the sub-harmonic signal. The pressure ramping-up phase required pressure overshoot. As illustrated in FIG. 25B, the correlation coefficient (R²) between the SC dose calculated by the area-under-the-curve of the SC level in the ramping-up phase and the fluorescence intensity of the delivered Evans blue was 0.992. As illustrated in FIG. 25C, the correlation coefficient (R²) between the SC dose calculated by the AUC of the SC level in the maintaining phase and the fluorescence intensity of the delivered Evans blue was 0.078. Comparing FIGS. 25B and 25C, the microbubble cavitation activity in the ramping-up phase, not the maintaining phase, was associated with the FUS-BBBO drug delivery outcome, The result suggested that the existing closed-loop control method could not reliably control the delivery outcome.

The results of these experiments demonstrated reliable and safe FUS-BBBO using the individualized closed-loop feedback control method disclosed herein at selected targeted cavitation levels. The use of FUS sonication at low pressure and short duration to establish the targeted cavitation level provided a strategy that accounted for individual differences in the detected cavitation signals and avoided overexposure. The disclosed feedback control algorithm had high stability and successfully controlled the FUS-BBBO drug delivery outcome. Optimal targeted cavitation levels for FUS-BBBO are influenced by both the performance of the cavitation controller and the delivery efficiency and safety. The results of these experiments highlighted the importance of controlling the FUS exposure to achieve efficient and safe BBBO.

### Example 8—Sonobiopsy of Glioblastoma-Derived Circulating Tumor DNA in Mouse GBM Model

To demonstrate the capability of focused ultrasound (FUS)-enabled liquid biopsy (sonobiopsy) in a mouse glioblastoma multiforme (GBM) model, the following experiments were conducted.

Human GBM cells (U87) with EGFRvIII overexpression (U87-EGFRvIII+) and carrying TERT C228T mutation were used to establish a mouse GBM model. U87-EGFRvIII+—ZsGreen+ cells, used for CTC detection, were generated by transduction of U87-EGFRvIII+ cells with the lentiviral construct pCRoatan that contained ZsGreen cDNA. Both cell lines were cultured as an adherent monolayer in DMEM supplemented with 10% fetal bovine serum, 2 mmol/L I-glutamine, and 100 units/mL penicillin. The cells were maintained at 37° C. in a humidified C02 (5%) atmosphere and the medium was changed as needed. Prior to implantation, cells were dispersed with a 0.05% solution of trypsin/EDTA and adjusted to concentrations needed for tumor implantation.

Immunodeficient mice (strain: NCI Athymic NCr-nu/nu, age: 6-8 weeks, Charles River Laboratory, Wilmington, MA, USA) were used to generate the xenograft GBM model. Briefly, mice were anesthetized and the head was fixed on a stereotactic device for injection of the tumor cells. Cells were injected and the tumor growth was monitored using a dedicated 4.7T small animal MRI system (Agilent/Varian DirectDrive™ console, Agilent Technologies, Santa Clara, CA, USA). Starting at 7 days and continuing every 3 days thereafter, MRI scans were acquired to monitor tumor growth and changes in neuroanatomy.

The mouse GBM model was used to detect EGFRvIII and TERT C228T mutations using sonobiopsy and using a conventional blood-based LBx (blood LBx) assay (control). Approximately 10-12 days after intracranial implantation, the mice were assigned to blood LBx (collect blood without FUS) or sonobiopsy (collect blood immediately after FUS).

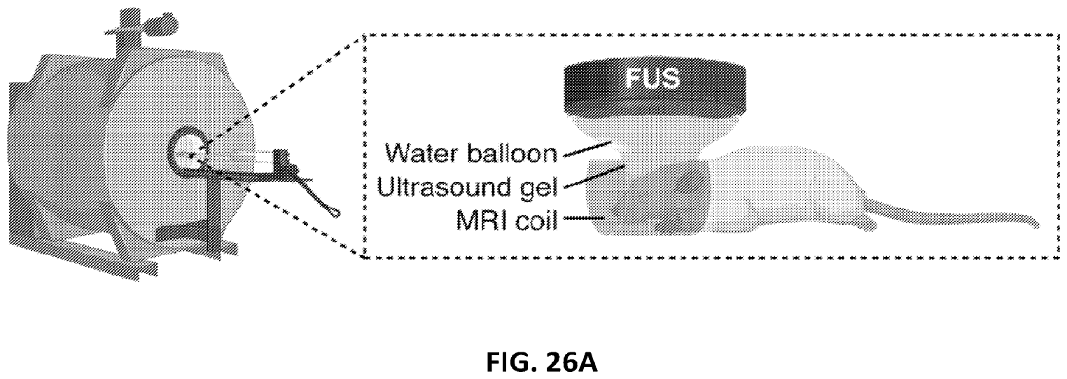

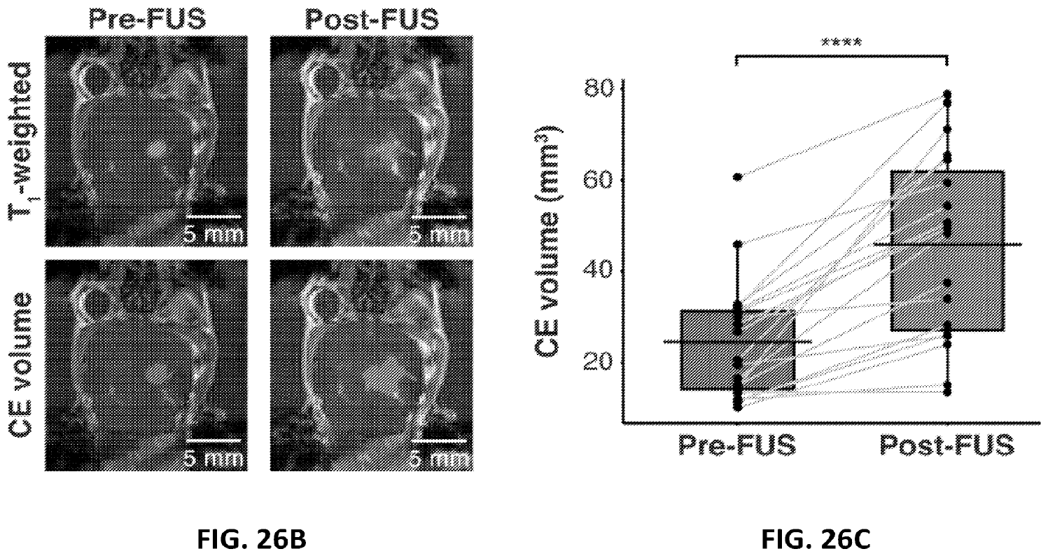

To implement sonobiopsy, a commercially available MRI-compatible FUS system (Image Guided Therapy, Pessac, France) was set up in a small animal MRI scanner (FIG. 26A). The system's MRI-compatible FUS transducer (Imasonics, Voray sur I'Ognon, France) was made of a 7-element annular array with a center frequency of 1.5 MHz, an aperture of 25 mm, and a radius of curvature of 20 mm. The axial and lateral full width at half maximums (FWHM) of the FUS transducer were 5.5 mm and 1.2 mm, respectively. Pressure values were derated to account for the 18% mouse skull attenuation. A catheter was placed in the mouse tail vein for intravenous injection of microbubbles as described below.

Coronal and axial T2-weighted MRI scans were acquired to image the mouse head and locate the geometrical focus of the transducer (same parameters as the aforementioned T2-weighted sequence used to monitor tumor growth). The MRI images were imported to a software program (ThermoGuide, Image Guided Therapy, Pessac, France) to locate the focus of the transducer via 3-point triangulation. The transducer was moved to the tumor center for FUS sonication. A pre-FUS axial T1-weighted MRI scan was performed to visualize the tumor-induced BBB permeability (same parameters as the aforementioned T1-weighted sequence used to monitor tumor growth) after intravenous injection of MR contrast agent gadoterate meglumine (Gd-DOTA; Dotarem, Guerbet, Aulnay sous Bois, France) at a dose of 1 mL/kg diluted 1:1 in 0.9% saline.

Definity microbubbles (Lantheus Medical Imaging, North Billerica, MA, USA) at a dose of 100 μL/kg were injected intravenously into the mice. FUS sonication started 15 seconds prior to microbubble intravenous injection (frequency: 1.5 MHz, pressure: 1.0 MPa, pulse repetition frequency: 5 Hz, duty cycle: 3.35%, pulse length: 6.7 ms, treatment duration: 3 min). FUS sonication was performed at 3 points, evenly spaced apart by 0.5 mm, to enable coverage of the entire tumor volume.

After sonication, Gd-DOTA was re-injected and a post-FUS axial T1-weighted MRI scan was performed (same parameters as pre-FUS T1-weighted sequence) to quantify the FUS-induced changes in BBB permeability.

The average tumor volumes for the blood LBx (n=21) group and the sonobiopsy groups (n=24) were not significantly different (p=0.78; unpaired two-sample Wilcoxon signed rank test) at 25.11±16.25 mm³ and 24.59±13.21 mm³, respectively. Contrast-enhanced (CE) T1-weighted MRI scans (FIG. 26B) were acquired to assess tumor growth and evaluate FUS-induced BBB disruption. FUS significantly increased the volume of tissue with enhanced BBB permeability by approximately 2-fold on average (FIG. 26C).

Terminal blood collection via cardiac puncture was performed 10 minutes after FUS sonication. Mouse whole blood (˜500 μL) was collected via cardiac puncture. Within 4 hours of collection, samples were centrifuged at 3000×g for 10 minutes at 4° C. to separate the plasma from the hematocrit. Plasma aliquots were put on dry ice immediately for snap freezing and stored at −80° C. subsequently for later downstream analysis.

A Plasma/Serum RNA/DNA Purification Mini Kit (Norgen Biotek, Thorold, ON, Canada) and a Plasma/Serum cfc-DNA/cfc-RNA Advanced Fractionation Kit (Norgen Biotek, Thorold, ON, Canada) were used to extract cfDNA from mouse plasma per manufacturer's protocol. cfDNA was eluted in 20 μL of each corresponding buffer and was quantified using Qubit Fluorometric Quantitation (Thermo Fisher Scientific, Carlsbad, CA, USA). The Bioanalyzer (Agilent Technologies, Palo Alto, CA, USA) was used to assess the size distribution and concentration of cfDNA extracted from plasma samples. The total cfDNA concentration was determined with the software as the area under the peaks in the mononucleosomal size range (140-230 bp).

An initial preamplification reaction was run prior to ddPCR in the case of very low DNA concentration. cfDNA was pre-amplified using Q5 hot start high-fidelity master mix (New England Biolabs, Beverly, MA, USA) with forward and reverse primer pairs for EGFRvIII and TERT C228T (same primers used for ctDNA analysis). Pre-amplification was performed with the Eppendorf Mastercycler: 98° C. for 3 min; 12 cycles of 98° C. for 30 s, 60° C. for 1 min; a final extension of 72° C. for 5 min, and 1 cycle at 4° C. infinite. Preamplified products were directly used for further ddPCR reactions.

EGFRvIII and TERT C228T were detected using custom sequence-specific primers and fluorescent probes. ddPCR reactions were prepared with 2×ddPCR Supermix for probes (no dUTP) (Bio-Rad, Hercules, CA, USA), 2 μL of target DNA product, 0.1 μM forward and reverse primers, and 0.1 μM probes. For the TERT C228T reaction mix, 100 μM 7-deaza-dGTP (New England Biolabs, Beverly, MA, USA) was added to improve PCR amplification of GC-rich regions in the TERT promoter. The QX200 manual droplet generator (Bio-Rad, Hercules, CA, USA) was used to generate droplets. The PCR step was performed on a C1000 Touch Thermal Cycler (Bio-Rad, Hercules, CA, USA) by use of the following program: 1 cycle at 95° C. for 10 min, 48 cycles at 95° C. for 30 s, 60° C. for 1 min, 1 cycle at 98° C. for 10 min, and 1 cycle at 12° C. for 30 min, 1 cycle at 4° C. infinite, all at a ramp rate of 2° C./s. All plasma samples were analyzed in technical duplicate or triplicate based on sample availability. Data were acquired on the QX200 droplet reader (Bio-Rad, Hercules, CA, USA) and analyzed using QuantaSoft Analysis Pro (Bio-Rad, Hercules, CA, USA). All results were manually reviewed for false positive and background noise droplets based on negative and positive control samples. Assays were considered positive if >3 droplets exceeded the threshold fluorescence. Otherwise, the specimen was determined to have 0 copies/μl. EGFRvIII and TERT C228T ctDNA concentrations (copies/μl plasma) were calculated by multiplying the concentration (provided by QuantaSoft) by elution volume, divided by the input plasma volume used during DNA extraction. A subject had a positive detection of the mutation when the levels of mutant ctDNA were >0 copies/μL. The EGFRvIII and TERT C228T sensitivities were calculated as the true positive rate, i.e., the number of true positives divided by the sum of true positives and false negatives. The 95% confidence intervals were calculated according to the familiar, asymptotic Gaussian approximation 1.96 √p(1−p)/n, where p represents sensitivity and n was the sample size.

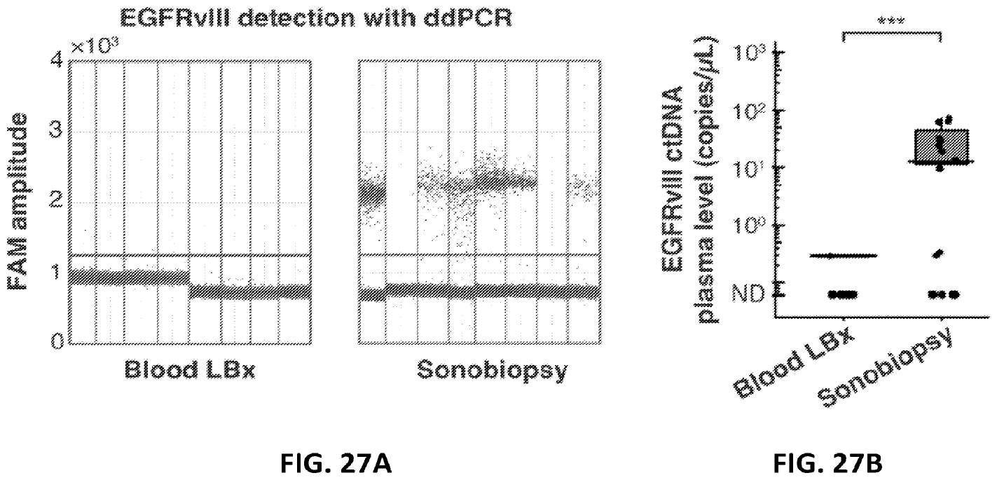

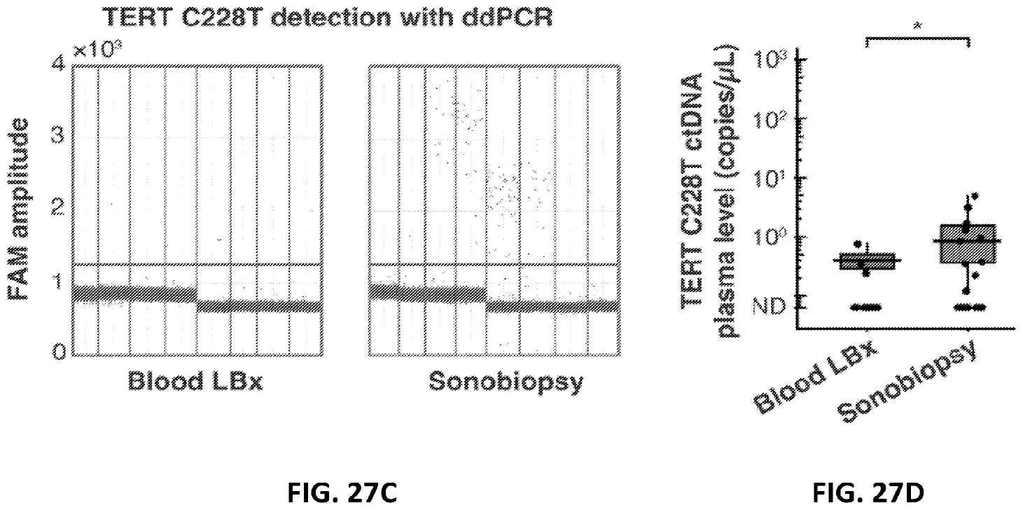

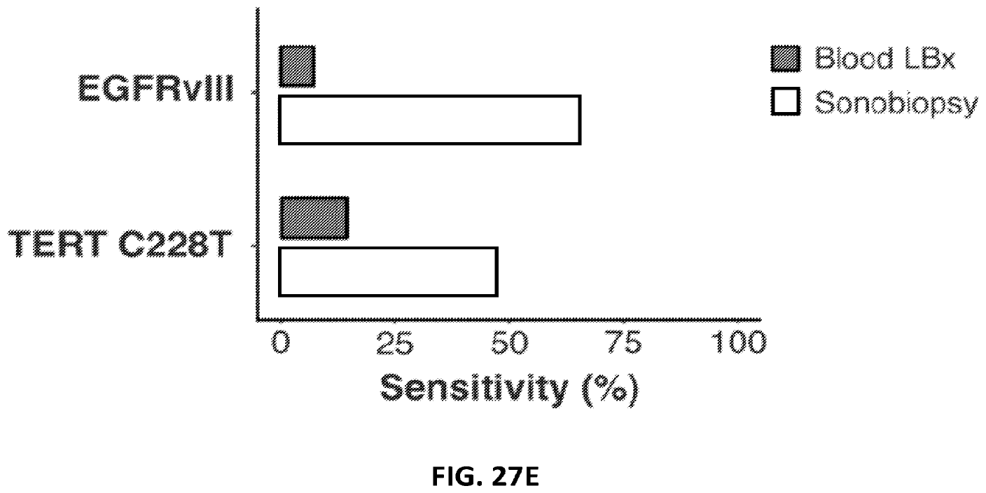

Analysis of the plasma cell-free DNA (cfDNA) found that sonobiopsy enhanced the release of cfDNA compared to conventional blood LBx. The plasma levels of mononucleosomal cfDNA (140-230 bp) increased approximately 2-fold with sonobiopsy. Custom ddPCR primers and probes for the detection of EGFRvIII and TERT C228T mutations were validated in vitro with cell lines that have known mutation statuses. The 1D amplitude plots showed the detection of EGFRvIII for 8 representative subjects in the blood LBx and sonobiopsy groups (FIG. 27A). The EGFRvIII ctDNA level in the sonobiopsy group was significantly greater (920-fold) than the blood LBx group (FIG. 27B). The 1D amplitude plots show the detection of TERT C228T for 8 representative subjects in the blood LBx and sonobiopsy groups (FIG. 27C). There was a significant increase (10-fold) in the levels of TERT C228T ctDNA with sonobiopsy compared with blood LBx (FIG. 27D). Sonobiopsy improved the diagnostic sensitivity from 7.14% to 64.71% for EGFRvIII and from 14.29% to 45.83% for TERT C228T (FIG. 27E). Taken together, sonobiopsy significantly enhanced the detection of brain tumor-specific mutations.

To assess the potential for tissue damage in the parenchyma associated with sonobiopsy, the following experiments were conducted. H&E staining was performed to quantify the extent of FUS-induced microhemorrhage and TUNEL staining was used to evaluate the number of apoptotic cells. After blood collection, mice were transcardially perfused with 0.01 M phosphate-buffered saline (PBS) followed by 4% paraformaldehyde. Brains were harvested and prepared for cryosectioning. The brains were horizontally sectioned into 15 μm slices and used for H&E staining to examine red blood cell extravasation and cellular injury or TUNEL staining to evaluate the number of apoptotic cells. The brain slices were digitally acquired with the Axio Scan.Z1 Slide Scanner (Zeiss, Oberkochen, Germany). QuPath v0.2.0 was used to detect areas of micro-hemorrhage and TUNEL expression. The imaged slice for mouse histological analysis was segmented into the tumor region of interest (ROI) that includes the tumor mass and extends 0.5 mm into its periphery, which is consistent with the safety objectives from previous studies and the potential damage caused by the external and lumen diameters of a biopsy needle. The parenchyma ROI was defined as the whole imaged slice without the tumor ROI. The tumor ROI for the histological analysis in pigs included the tumor mass and a 3 mm margin.

After color deconvolution (hematoxylin vs. eosin), areas of micro-hemorrhage were detected using the positive-pixel count algorithm. The micro-hemorrhage density was calculated as the percentage of positive pixel area over the total stained area in the respective ROI. The number of apoptotic cells was detected using the positive cell detection algorithm. The TUNEL density was calculated as the percentage of positive cells over the total stained cells in the respective ROI.

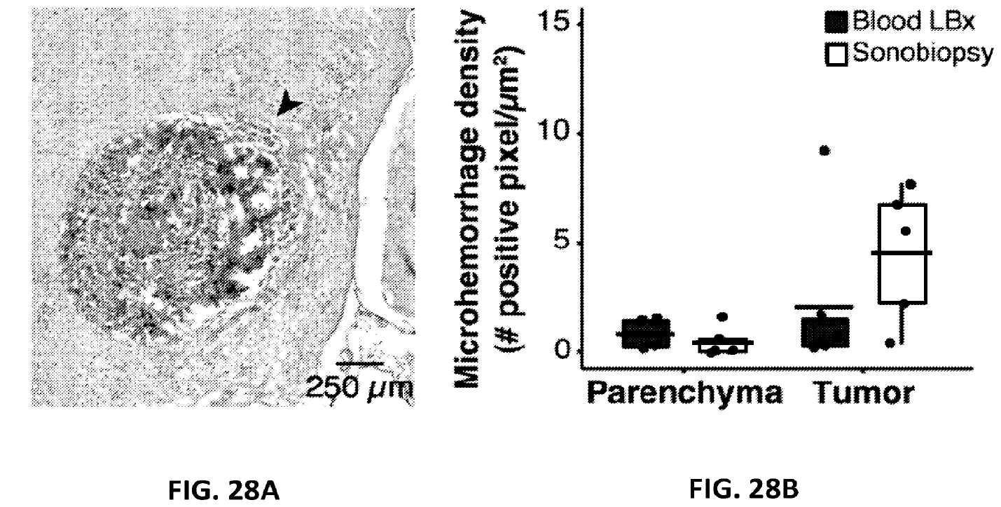

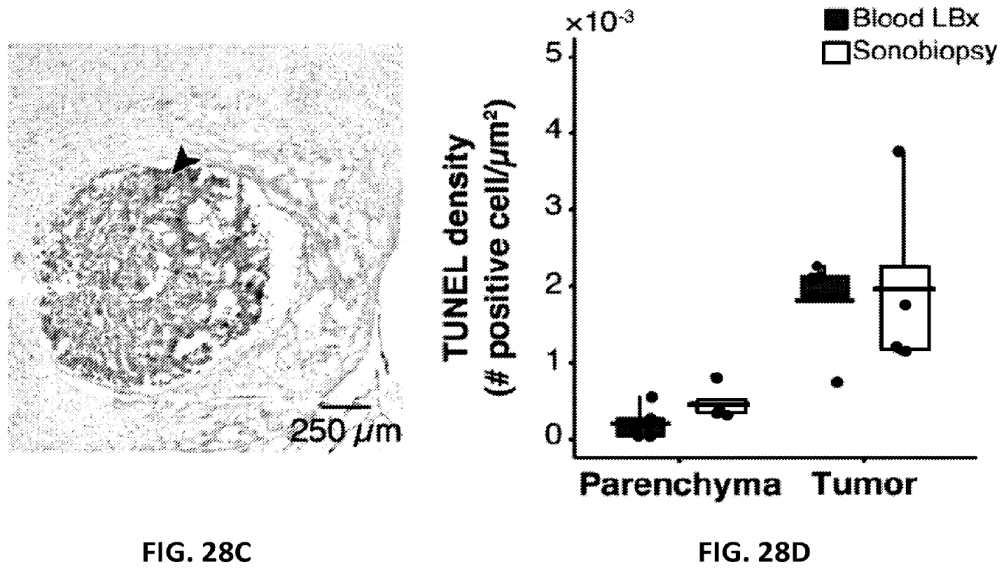

Sonobiopsy led to a non-significant increase in detected microhemorrhages within the tumor region of interest (ROI) (FIGS. 28A and 28B), and no off-target damage was detected in the brain parenchyma. Sonobiopsy also did not change the TUNEL expression in the tumor ROI or the brain parenchyma (FIGS. 28C and 28D).

The results of these experiments demonstrated the safety and effectiveness of focused ultrasound (FUS)-enabled liquid biopsy (sonobiopsy) in the mouse glioblastoma multiforme (GBM) model.

### Example 9—Sonobiopsy of Glioblastoma-Derived Circulating Tumor DNA in Porcine GBM Model

To demonstrate the capability of focused ultrasound (FUS)-enabled liquid biopsy (sonobiopsy) in a porcine glioblastoma multiforme (GBM) model, the following experiments were conducted.

A porcine model of GBM was developed that included bilateral implantation of the U87-EGFRvIII+ cells described in Ex. 8 in the pig cortex followed by immunosuppressant treatment to prevent rejection of the grafted cells. Approximately 3×10⁶ cells for each tumor were implanted in pigs.

Pigs (breed: Yorkshire white, age: 4 weeks, sex: male, weight: 15 lbs., Oak Hill Genetics, Ewing, IL, USA) were implanted with the tumor cells on day 0 with an established protocol. After the pig was sedated, the head was shaved, prepared for sterile surgery, and immobilized in a stereotactic frame on the operating table. The bite bar and ear bars were positioned to secure the head such that the top of the skull was level with the operating table. A 2-3 cm midline cranial skin incision was made and two 5 mm burr holes were drilled 5 mm posterior from bregma and 7 mm to the subject's right and left from midline without breaking the dura (Dremel, Racine, WI, USA). A 50 μL syringe (Hamilton, Reno, NV, USA) used for tumor cell injection was fixed on the stereotactic frame and positioned in the burr hole with the tip at the dura. The syringe was lowered 9 mm to the injection site and the Micro4 controller (World Precision Instruments, Sarasota, FL, USA) infused 40 μL with a rate of 44 nL/sec. There was a 5-minute delay between infusion completion and needle withdrawal to allow the cells to settle in the tissue and prevent backflow. The burr holes were filled with gel foam and the skin incisions were closed with two layers of sutures. A cyclosporine oral solution (Neoral, Novartis Pharmaceuticals, East Hanover, NJ, USA) was administered (25 mg/kg) twice daily via gavage.

Seven days post-surgery, a contrast-enhanced sagittal T1-weighted gradient echo MRI scan (TR/TE: 23/3.03 ms; slice thickness: 0.9 mm; in-plane resolution: 0.94×0.94 mm2; matrix size: 192×192; flip angle: 27°) was acquired on the 3T Siemens PRISMA Fit clinical scanner (Siemens Medical Solutions, Malvern, PA, USA) to validate tumor growth. An intravenous catheter was placed in the ear for ease of microbubble and gadolinium injections. During the treatment and MR scans, a pulse oximeter (Nonin 7500FO, Plymouth, MN, USA) monitored blood oxygen levels and pulse rate, while heated blankets were used to regulate the temperature.

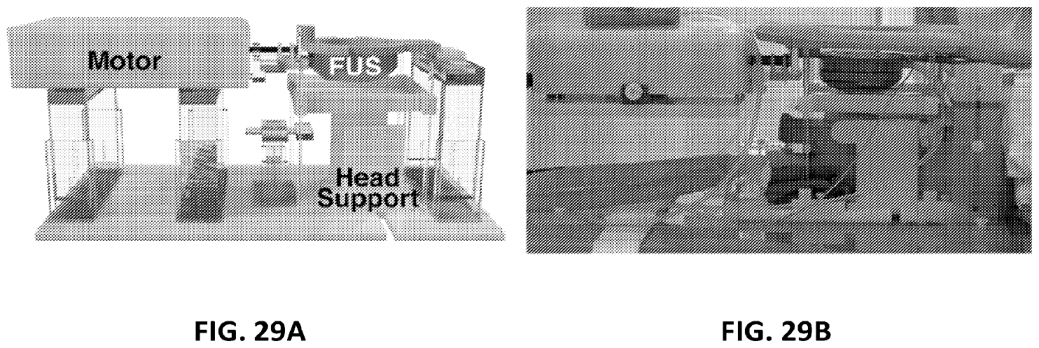

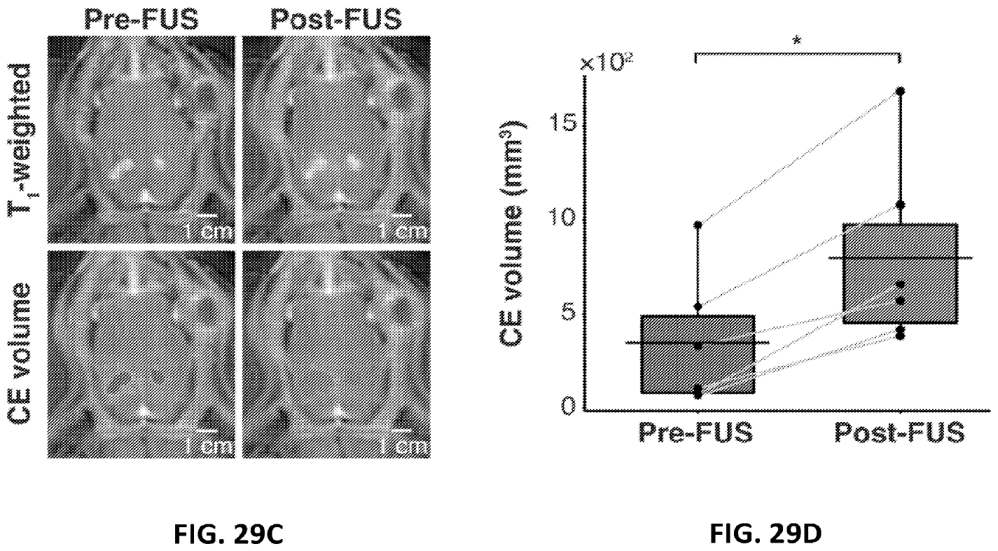

The bilateral tumor model capitalized on the unique feature of the large brain volume in pigs and provided the opportunity for sonobiopsy to target two distinct targets in individual pigs. Sonobiopsy was performed approximately 11 days after intracranial implantation. A customized MRI-guided FUS device was developed to sonicate each large animal tumor sequentially (1-hour delay to minimize cross-contamination from biomarker release of the first sonication) in a clinical MRI scanner (FIGS. 29A and 29B).

A customized MRI-guided FUS device and an established FUS procedure were used for successful BBB disruption. The pig head was fixed in a stereotactic head frame with a bite bar and head supports and coupled with the transducer. The FUS system (Image Guided Therapy, Pessac, France) included an MR-compatible 15-element transducer with a center frequency of 650 kHz, an aperture of 65 mm, a radius of curvature of 65 mm, and an adjustable coupling bladder. The FUS system was attached to an MR-compatible motor for enhanced targeting precision. The FUS transducer calibration is provided in the supplementary information. Briefly, the in vivo acoustic pressure was estimated with the top portion of a harvested ex vivo pig skull. The axial and lateral FWHM of the transducer was 3.0 mm and 20.0 mm, respectively.

FUS was performed under MR guidance of the 1.5T Philips Ingenia clinical MR scanner (Philips Medical Systems, Inc., Cleveland, OH, USA). Coronal and axial T2-weighted spin-echo MR images were acquired to examine the neuroanatomy for treatment planning (TR/TE: 1300/130 ms; slice thickness: 1.2 mm; in-plane resolution: 0.58×0.58 mm2; matrix size: 448×448; flip angle: 90°). Coronal and axial T2*-weighted gradient echo MR scans were used to visualize the presence of air bubbles in the acoustic coupling media (TR/TE: 710/23 ms; slice thickness: 2.5 mm; in-plane resolution: 0.98×0.98 mm2; matrix size: 224×224; flip angle 18°). The FUS targeting was performed with the same ThermoGuide workflow as the mouse sonobiopsy as described in EX. 8. Gadobenate dimeglumine (Gd-BOPTA; Multihance, Bracco Diagnostics Inc., Monroe Township, NJ, USA) was intravenously injected at a dose of 0.2 mL/kg and an axial T1-weighted ultrafast spoiled gradient echo MR scan was acquired as a pre-FUS baseline (TR/TE: 5/2 ms; slice thickness: 1.5 mm; in-plane resolution: 0.68×0.68 mm2; matrix size: 320×320; flip angle 10°).

Definity microbubbles (Lantheus Medical Imaging, North Billerica, MA, USA) at a dose of 20 μL/kg were injected intravenously. FUS sonication started 15 seconds prior to microbubble intravenous injection using the following parameters: frequency: 0.65 MHz, pressure: 3.0 MPa (measured in water; 2.0 MPa measured with the ex vivo pig skull), pulse repetition frequency: 1 Hz, duty cycle: 1%, pulse length: 10 ms, treatment duration: 3 min. The bolus injection was determined by the precedence set by the clinical papers that have a similar injection paradigm and the observation that the contrast enhancement via bolus is greater than the enhancement via infusion. The 3-minute sonication was previously determined as the time point when all the microbubbles were depleted, as observed by a lack of stable cavitation during passive cavitation detection. The treatment was repeated at 4 individual points spaced 3 mm apart to ensure coverage of the tumor.

After FUS sonication was completed, Gd-BOPTA was intravenously injected and an axial T1-weighted MR scan was acquired (same parameters as the pre-FUS T1-weighted sequence) to assess the BBB permeability. Coronal T2*-weighted images were acquired (same parameters as pre-FUS) to assess the potential for FUS-induced tissue damage.

Contrast-enhanced T1-weighted MRI scans confirmed successful BBB disruption of both tumors (FIG. 29C), where the total CE volume significantly increased post-FUS (FIG. 29D).

Blood samples (5 mL) were collected immediately before and 10 minutes after FUS sonication of each tumor. Pig whole blood (˜10 mL) was collected via percutaneous catheter within a peripheral vessel using BD Vacutainer K2 EDTA tubes (Becton Dickinson, Franklin Lakes, NJ, USA). Plasma aliquots were isolated and stored as described in Ex. 8. Circulating tumor DNA was extracted and quantified using ddPCR as described in Ex. 8.

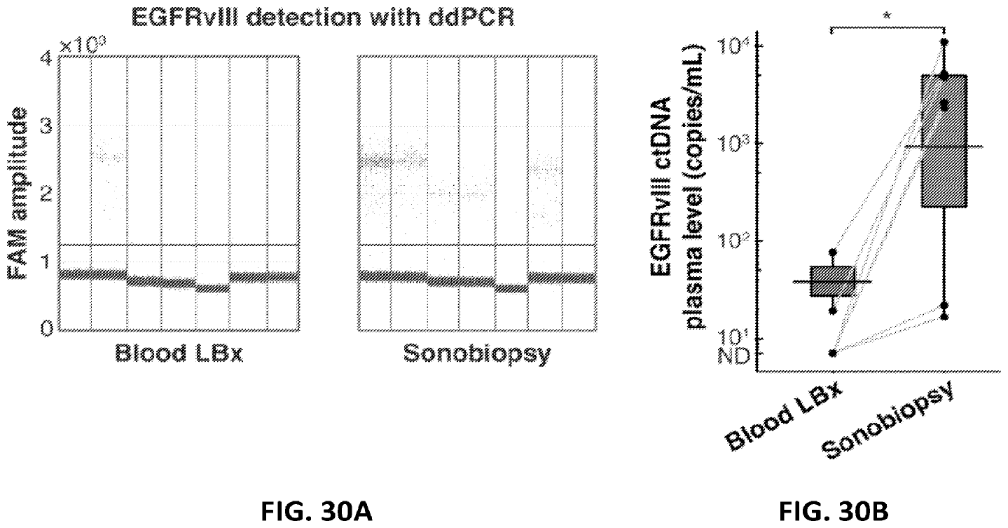

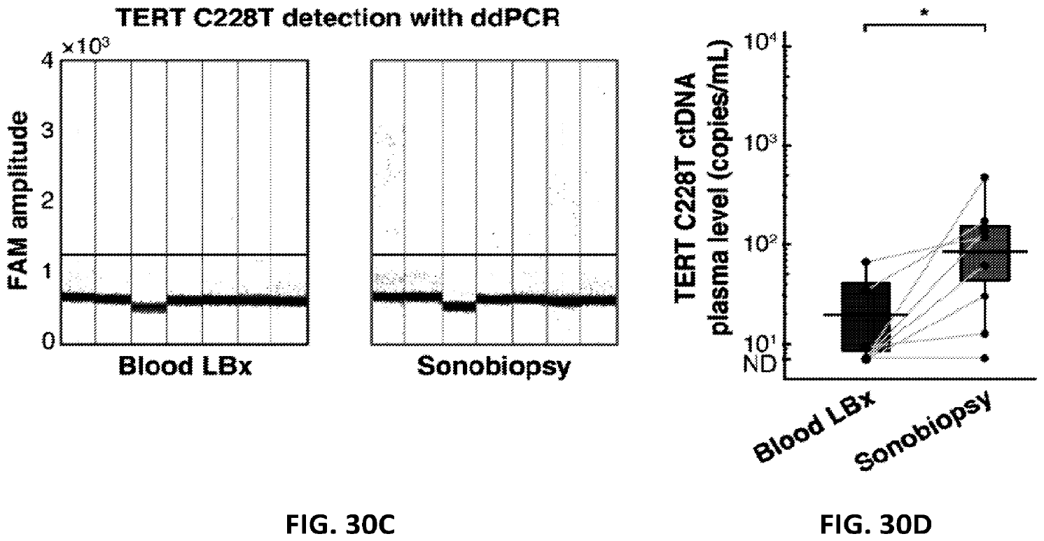

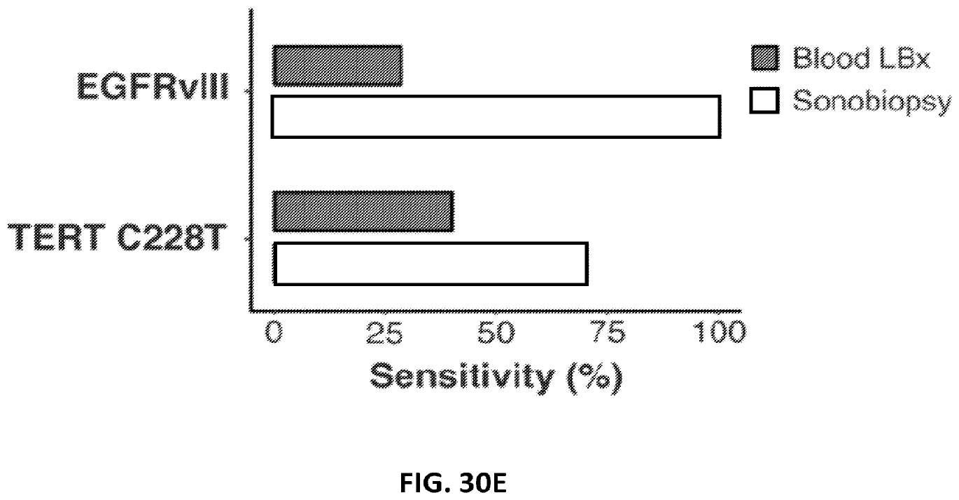

The ddPCR 1D amplitude plots demonstrate the detection of EGFRvIII for all subjects in the blood LBx (pre-FUS) and sonobiopsy (post-FUS) groups (FIG. 30A). Sonobiopsy significantly enhanced the release of EGFRvIII ctDNA into the blood by 270-fold (FIG. 30B). The 1D fluorescence amplitude plots show the detection of TERT C228T with ddPCR for all subjects in the blood LBx and sonobiopsy groups (FIG. 30C). The levels of TERT C228T ctDNA significantly increased 9-fold with sonobiopsy (FIG. 30D). The sonobiopsy-induced release improved the diagnostic sensitivity from 28.57% to 100% for EGFRvIII and from 42.86% to 71.43% for TERT C228T (FIG. 30E). Sonobiopsy was shown to significantly enhance the detection of brain tumor-specific mutations in a pig GBM model.

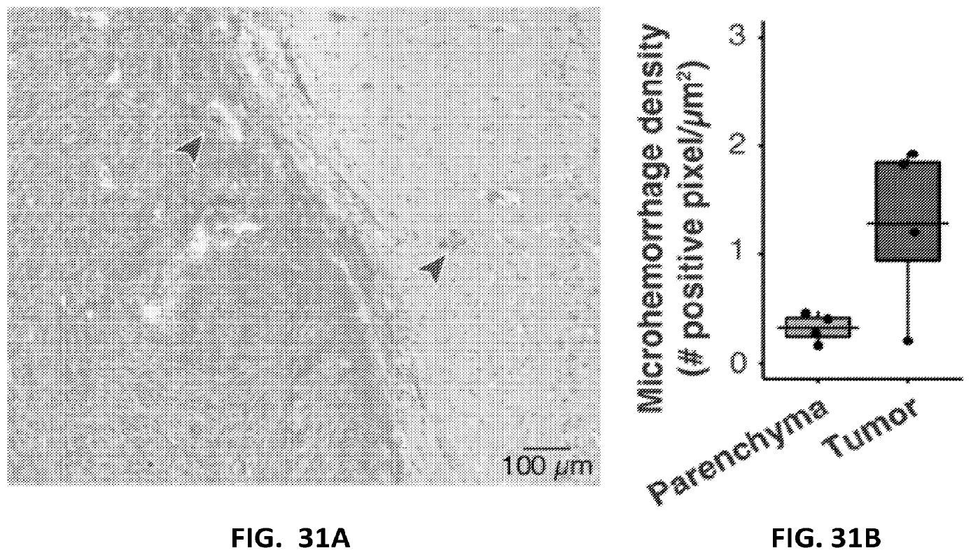

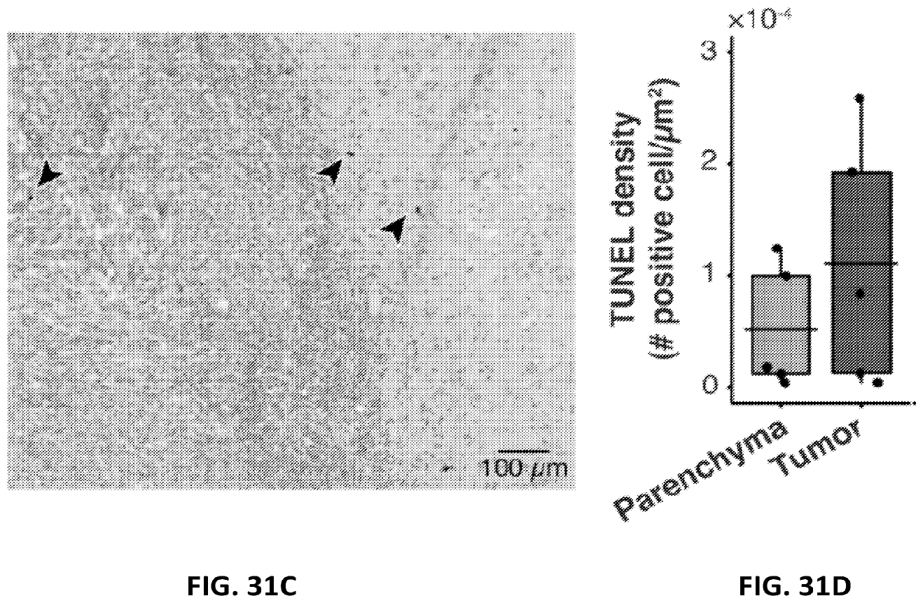

To evaluate the safety of large animal sonobiopsy, the following experiments were conducted. Pig brains were harvested and fixed in 10% formalin. Histological staining with H&E and TUNE was performed as described in EX. 8 to detect microhemorrhaging associated with sonobiopsy. H&E staining showed the presence of microhemorrhage near the edge of the tumors in some cases (FIG. 31A). However, there was no significant difference in microhemorrhage density between the sonicated tumor ROI and the unsonicated parenchyma (FIG. 31B). In addition, the TUNEL staining (FIG. 31C) suggested there was no significant difference between the number of apoptotic cells in the parenchyma compared with the tumor ROI (FIG. 31D). MRI was used to evaluate acute tissue damage post-FUS. Abnormalities in the post-FUS T2*-weighted images, i.e., signal intensity changes, were observed. The observed tissue damage was consistent with the reversible damage observed in clinical trials of FUS-induced BBB disruption for brain drug delivery.

The results of these experiments demonstrated the safety and effectiveness of focused ultrasound (FUS)-enabled liquid biopsy (sonobiopsy) in the porcine glioblastoma multiforme (GBM) model.

What is claimed is:

1. A method for performing a liquid biopsy to diagnose a brain disorder of a subject, the method comprising:
a. injecting an amount of microbubbles into the subject;
b. opening a blood-brain barrier of the subject using a focused ultrasound blood-brain barrier opening (FUS-BBBO) device to release at least one biomarker from a brain of the subject into blood of the subject;
c. obtaining a biological sample comprising the at least one biomarker, the biological sample comprising a blood sample or a CSF sample from the subject; and
d. diagnosing the brain disorder based on the at least one biomarker isolated from the biological sample.

2. The method of claim 1, wherein opening the blood-brain barrier using the FUS-BBBO device further comprises:
a. sonicating the brain of the subject at a baseline sonication pressure and detecting a baseline stable cavitation level from the subject using the FUS-BBBO device, the baseline stable cavitation levels falling above signal noise and below a stable cavitation level sufficient to induce BBBO, wherein the subject is injected with the amount of microbubbles prior to sonication;
b. sonicating the subject at a series of stepwise increasing sonication pressures and detecting a corresponding series of cavitation levels until a target cavitation level (TCL) is detected, wherein the target cavitation level is a predetermined amount above the baseline stable cavitation level; and
c. continuously sonicating the subject to maintain the TCL to induce BBBO in the subject.

3. The method of claim 2, wherein detecting the baseline cavitation levels, the series of cavitation levels, and the target cavitation levels further comprises detecting microbubble cavitation signals, wherein the microbubble cavitation signals are produced by microbubbles in response to sonication by the FUS-BBBO device.

4. The method of claim 3, wherein the microbubble cavitation signals are processed using a Fast-Fourier transform (FFT) algorithm to produce the baseline cavitation levels, cavitation levels, and TCL.

5. The method of claim 2, wherein the target cavitation level comprises one of 0.5 dB, 1 dB, 2 dB, 3 dB, or 4 dB above the baseline stable cavitation level.

6. A system to control operation of a focused ultrasound blood-brain barrier opening (FUS-BBBO) device configured to perform FUS-BBBO on a subject, the system comprising a computing device operatively coupled to the FUS-BBBO device, the computing device comprising at least one processor, the at least one processor configured to:
a. sonicate the subject at a baseline sonication pressure and detect a baseline stable cavitation level from the subject using the FUS-BBBO device, the baseline stable cavitation levels falling above signal noise and below a stable cavitation level sufficient to induce BBBO, wherein the subject is injected with microbubbles prior to sonication;
b. sonicate the subject at a series of stepwise increasing sonication pressures and detecting a corresponding series of cavitation levels until a target cavitation level (TCL) is detected, wherein the target cavitation level is a predetermined amount above the baseline stable cavitation level; and
c. continuously sonicate the subject to maintain the TCL to induce BBBO in the subject.

7. The system of claim 6, further comprising at least one passive cavitation detection (PCD) transducer to detect the baseline cavitation levels, the series of cavitation levels, and the target cavitation levels.

8. The system of claim 6, wherein detecting the baseline cavitation levels, the series of cavitation levels, and the target cavitation levels further comprises detecting microbubble cavitation signals, wherein the microbubble cavitation signals are produced by microbubbles in response to sonication by the FUS-BBBO device.

9. The system of claim 8, wherein the microbubble cavitation signals are processed using a Fast-Fourier transform (FFT) algorithm to produce the baseline cavitation levels, cavitation levels, and TCL.

10. The system of claim 8, wherein the microbubble cavitation signals comprise acoustic signals with a frequency within a bandwidth of a center frequency of the PCD transducer.

11. The system of claim 6, wherein the target cavitation level comprises one of 0.5 dB, 1 dB, 2 dB, 3 dB, or 4 dB above the baseline stable cavitation level.

12. A method of performing FUS-BBBO on a subject, the method comprising:
a. injecting the subject with microbubbles;
b. sonicating the subject at a baseline sonication pressure and detecting a baseline stable cavitation level from the subject after injection of microbubbles using the FUS-BBBO device, the baseline stable cavitation levels falling above signal noise and below a stable cavitation level sufficient to induce BBBO;
c. sonicating the subject at a series of stepwise increasing sonication pressures and detecting a corresponding series of cavitation levels until a target cavitation level (TCL) is detected, wherein the target cavitation level is a predetermined amount above the baseline stable cavitation level; and
d. continuously sonicating the subject to maintain the TCL to induce BBBO in the subject.

13. The method of claim 12, wherein detecting the baseline cavitation levels, the series of cavitation levels, and the target cavitation levels are performed using at least one passive cavitation detection (PCD) transducer.

14. The method of claim 12, wherein detecting the baseline cavitation levels, the series of cavitation levels, and the target cavitation levels further comprises detecting microbubble cavitation signals, wherein the microbubble cavitation signals are produced by microbubbles in response to sonication by the FUS-BBBO device.

15. The method of claim 14, wherein the microbubble cavitation signals are processed using a Fast-Fourier transform (FFT) algorithm to produce the baseline cavitation levels, cavitation levels, and TCL.

16. The method of claim 14, wherein the microbubble cavitation signals comprise acoustic signals with a frequency within a bandwidth of a center frequency of the PCD transducer.

17. The method of claim 12, wherein the target cavitation level comprises one of 0.5 dB, 1 dB, 2 dB, 3 dB, and 4 dB above the baseline stable cavitation level.

18. A device for transcranial cavitation localization in a subject, the device comprising:
a. four acoustic sensors to detect cavitation signals within a skull of the subject, the four acoustic sensors comprising S1, S2, S3, and S4, the four acoustic sensors positioned in a fixed pattern configured to conform to the skull of the subject;
b. a focused ultrasound (FUS) transducer to sonicate a volume of interest within the skull of the subject; and
c. a computing device comprising at least one processor, the at least one processor configured to:
i. sonicate the volume of interest using the FUS transducer;
ii. receive a plurality of cavitation signals from within the skull of the subject at the four acoustic sensors, wherein the subject is injected with microbubbles;
iii. identify at least three time delays based on the plurality of cavitation signals, the at least three time delays comprising a difference in an arrival time of a cavitation signal at one of acoustic sensors S1, S2, S3, and S4 relative to one of the remaining acoustic sensors; and
iv. localize the cavitation signal source based on the at least three time delays.

19. The device of claim 18, wherein the four acoustic sensors are positioned in a hemispherical pattern.

20. The device of claim 18, wherein the four acoustic sensors are positioned with three acoustic sensors arranged along a circumference of a circle and one acoustic sensor positioned within the circle and perpendicularly offset from the plane of the circle.

21. The device of claim 18, wherein each time delay of the at least three time delays is identified based on the maximum cross-correlation of a first sample of cavitation signals detected at a first acoustic detector and a second sample of cavitation signals detected at a second acoustic detector.

22. The device of claim 18, wherein the cavitation signal source is localized using a time difference of arrival (TDOA) method.
